(19) United States
(12) Patent Application Publication
(10) Pub. No.: US 2010/0239620 A1
(43) Pub. Date: Sep. 23, 2010

(54) MONODISPERSE PARTICLES

(76) Inventors: Michael Francis Butler, Sharnbrook (GB); Ramin Djalali, Warwickshire (GB)

Correspondence Address: DAVIS WRIGHT TREMAINE LLP/Los Angeles, 865 FIGUEROA STREET, SUITE 2400, LOS ANGELES, CA, 90017-2566, US

(21) Appl. No.: 12/600,526

(22) PCT Filed: May 13, 2008
(86) PCT No.: PCT/EP08/55823

§ 371 Date: Jun. 8, 2010

(30) Foreign Application Priority Data
May 18, 2007 (IB) PCT/IB2007/001694

(51) Int. Cl. A61K 8/02; A61K 8/81; A61K 8/88; A61K 8/85; A61K 8/87; A61K 8/86; A61Q 1/06; A61Q 1/10; A61Q 1/08; C09D 11/02; A61K 8/00; A61Q 5/02

(52) U.S. Cl. 424/401; 424/64; 424/63; 424/70.7; 424/61; 106/31.13; 510/119

(57) ABSTRACT

The present invention relates to novel monodisperse particles, which comprise at least one broad spectrum absorber contrast agent and/or at least one precursor of a broad spectrum absorber contrast agent, which is encapsulated in the monodisperse particle, novel colourant compositions, to uses thereof in colouring a substrate and to substrates that are coloured (at least in part) using the colourant compositions.

### FIELD OF THE INVENTION

The present invention relates to novel monodisperse particles, which comprise a low amount of at least one broad spectrum absorber contrast agent and/or at least one precursor of a broad spectrum absorber contrast agent, which is encapsulated in the monodisperse particle, novel colourant compositions, to uses thereof in colouring a substrate and to substrates that are coloured (at least in part) using the colourant compositions.

### BACKGROUND TO THE INVENTION

Chromophores are materials that impart a colour. Colours typically are produced by the absorption or emission of light and the loss of photon energy.

Colours may also be produced by structural effects, for example when light is reflected and/or diffracted by highly regular structures having dimensions that match the wavelength of visible light. Such structures can have different colours when viewed from different angles, which effect is known in the art as “structural colour”. Examples of colours produced in this way can be found in nature, for example in some butterfly wings, peacock feathers, the hair of a seamouse and semi-precious gemstones such as opals.

Compositions for producing colours by structural effects are known. For example, U.S. Pat. No. 6,756,115 describes a particle matrix with opalescent effect comprising monodisperse spheres and one or more types of smaller colloidal particles occupying partially or completely the empty spaces between the monodisperse spheres, which smaller colloidal particles adjust the refractive index ratio between the spheres and the media between them to achieve a predetermined colour effect and bind the matrix together.

Müller et al. (Chem. Mater. 2000, 12, 2508-2512) describes dye-impregnated poly(methylmethacrylate) opaline photonic films that demonstrate incomplete photonic band gap structure and related modification of dye photoluminescence spectrum. The dye used in Müller et al. is a fluorescent dye, which does not absorb visible light.

WO2005/063902 describes an ink jet composition that comprises a carrier liquid and monodisperse particles. The monodisperse particles form 3D photonic crystals upon application to a substrate surface by arranging in a 3D, tightly packed, regular and spherical structure on the substrate surface. In one example of WO2005/063902, a black ink is printed onto glossy paper or avery vinyl and then overprinted with an ink that comprises polymethylmethacrylate particles (but no dye/pigment).

U.S. Pat. No. 6,818,051 describes particles having an opalescent effect. The particles comprise monodisperse spheres in a 3D, regularly ordered structure that is closely packed in terms of domains and is mechanically stabilised by physical or chemical modification.

U.S. Pat. No. 6,337,131 describes core shell particles, whose core and shell are capable of forming a two-phase system with the shell material being filmable and the core being essentially form-stable under shell-filming conditions and being only minimally swellable by the shell material. The materials of the core and shell have a specified refractive index difference. U.S. Pat. No. 6,337,131 refers to obtaining an impressive angle-dependent colour change when the core shell particles are applied to a dark substrate.

U.S. Pat. No. 6,894,086 discloses colourants comprising an ordered periodic array of particles held in a matrix, wherein the matrix and the particles have a specified difference in refractive index. In one example of U.S. Pat. No. 6,894,086, the colourant was painted on to a black basecoat.

None of the aforementioned prior art documents disclose a colourant composition that comprises a broad spectrum absorber contrast agent as defined in this specification, i.e. that absorbs substantially all of the light having a wavelength in a range that corresponds to the wavelength of visible light and/or that is organic.

### SUMMARY OF THE INVENTION

The present inventors have now provided novel monodisperse particles, which comprise a low amount of at least one broad spectrum absorber contrast agent and/or at least one precursor of a broad spectrum absorber contrast agent, which is encapsulated in the monodisperse particle. Such encapsulated monodisperse particles result in colourant compositions that produce enhanced structural colour effects, for example compared to the prior art compositions discussed above.

The monodisperse particles of the present invention are easy to produce. Additionally, there are easy to handle and even with less ordered crystals formed from monodisperse particles the colour effect is brilliant.

According to one aspect of the present invention, there are provided monodisperse particles capable of forming a colloidal crystal, wherein at least one broad spectrum absorber contrast agent and/or at least one precursor of a broad spectrum absorber contrast agent is encapsulated in the particles, characterised in that the monodisperse particles comprise
(i) 97-99.999 wt-%, based on the total weight of the monodisperse particles, of the monodisperse particle material, and
(ii) 0.0001-3 wt-%, based on the total weight of the monodisperse particles, of at least one broad spectrum absorber contrast agent and/or at least one precursor of a broad spectrum absorber contrast agent, which is encapsulated.

The goal of the present invention is not to produce coloured particles, but to produce colourless particles, wherein a colour effect is achieved by colloidal crystal arrangement. Therefore the amount of at least one broad spectrum absorber contrast agent and/or at least one precursor of a broad spectrum absorber contrast agent encapsulated in such monodisperse particles must not be too high (less than 3 wt-%, based on the total weight of the monodisperse particles). The amount varies depending on the absorption coefficient of the broad spectrum absorber contrast agent and/or the precursor of a broad spectrum absorber contrast agent.

The weight percentages are based on the total weight of the monodisperse particles which comprise the broad spectrum absorber contrast agent and/or at least one precursor of a broad spectrum absorber contrast agent. Therefore it is obvious that the addition of the wt-% of (i) and of (ia) is 100%.

According to a further aspect of the present invention, there are provided monodisperse particles capable of forming a colloidal crystal, wherein at least one broad spectrum absorber contrast agent and/or at least one precursor of a broad spectrum absorber contrast agent is encapsulated in the particles, characterised in that the monodisperse particles comprise
(i) 99-99.999 wt-%, preferably 99.5-99.9999, based on the total weight of the monodisperse particles, of the monodisperse particle material, and
(ii) 0.0001-1 wt-%, preferably 0.0001-0.5 wt-%, based on the total weight of the monodisperse particles, of at least one broad spectrum absorber contrast agent and/or at least one precursor of a broad spectrum absorber contrast agent, which is encapsulated.

A further embodiment relates to monodisperse particles capable of forming a colloidal crystal, wherein at least one broad spectrum absorber contrast agent and/or at least one precursor of a broad spectrum absorber contrast agent is encapsulated in the particles, characterised in that the monodisperse particles comprise
(i) 97-99.9999 wt-%, preferably 99-99.999 wt-%, more preferably 99.5-99.9999, based on the total weight of the monodisperse particles, of the monodisperse particle material, and
(ii) 0.0001-3 wt-%, preferably 0.0001-1 wt-%, more preferably 0.0001-0.5 wt-%, based on the total weight of the monodisperse particles, of at least one broad spectrum absorber contrast agent and/or at least one precursor of a broad spectrum absorber contrast agent, which is encapsulated,
   with the proviso that example 1 of PCT Application No PCT/EP2006/010882 is excluded from the scope of protection.

A further embodiment relates to monodisperse particles capable of forming a colloidal crystal, wherein at least one broad spectrum absorber contrast agent and/or at least one precursor of a broad spectrum absorber contrast agent is encapsulated in the particles, characterised in that the monodisperse particles comprise
(i) 97-99.9999 wt-%, preferably 99-99.999 wt-%, more preferably 99.5-99.9999, based on the total weight of the monodisperse particles, of the monodisperse particle material, and
   (ii) 0.0001-3 wt-%, preferably 0.0001-1 wt-%, more preferably 0.0001-0.5 wt-%, based on the total weight of the monodisperse particles, of at least one broad spectrum absorber contrast agent and/or at least one precursor of a broad spectrum absorber contrast agent, which is encapsulated,
      with the proviso that the encapsulated monodisperse particles are not consisting of methylmethacrylate and Alizarin Blue Black.

That means that mixtures of broad spectrum absorber contrast agents and their precursors containing Alizarin Blue black are not excluded from the scope of protection.

Alizarin Blue Black is C.I. Mordant Black 13 (C.I. 63615).

By the term “monodisperse particle material” it is meant the material which encapsulates the broad spectrum absorber contrast agent and/or the precursor of a broad spectrum absorber contrast agent and forms the monodisperse particles.

The monodisperse particles may be formed from the same material or they may also be formed from different materials.

By the term “monodisperse particles” we mean particles wherein at least 60% of the particles fall within a specified particle size range. For example, the monodisperse particles preferably have a diameter that deviates less than 10% in root mean square (rms), more preferably that deviates less than 5% in rms diameter.

By the term “encapsulated” we mean that the broad spectrum absorber contrast agent and/or at least one precursor of a broad spectrum absorber contrast agent is enclosed or embedded within the monodisperse particles. The broad spectrum absorber contrast agent and/or at least one precursor of a broad spectrum absorber contrast agent is (more or less) evenly distributed in the monodisperse particles. That means that it is not concentrated mainly in one part of the monodisperse particle.

Typically, the monodisperse particles are capable of forming a colloidal crystal that reflects and/or diffracts light having a wavelength in a range that corresponds to the wavelength of visible light.

Suitable monodisperse particles typically have a rms diameter of less than about 1 μm and greater than about 1 nm, and are, therefore, classed as “nanoparticles”. In particular, the monodisperse particles may have a rms diameter of greater than 100, preferably greater than 120 nm. Preferably the monodisperse particles may have a rms diameter of less than 900 nm, preferably less than 800 nm. More preferably, the rms diameter of the monodisperse particles is in the range of from 120 nm to 800 nm.

Monodisperse particles suitable for use in the colourant compositions of the present invention may be of varying geometry. They can have any geometrical forms. For example, the monodisperse particles may be shaped as needles, plates and/or rods and/or may be substantially spherical. The shape of the monodisperse particles does not significantly influence their effectiveness in the colourant compositions of the present invention.

The monodisperse particles according to the present invention are no core/shell particles. A core/shell particle consists of (at least) two layers, the core and the shell, whereas the particles of the present invention only consist of one layer. The broad spectrum absorber contrast agent and/or at least one precursor of a broad spectrum absorber contrast agent is not concentrated in the centre of the monodisperse particle (or in another part of the monodisperse particle). It is distributed in the whole monodisperse particle. Usually it is evenly distributed therein.

Therefore a further embodiment of the present invention relates to monodisperse particles, wherein at least one broad spectrum absorber is encapsulated and where the broad spectrum absorber contrast is distributed over the whole monodisperse particle

In one aspect of the present invention, the monodisperse particles are substantially spherical. By the term “substantially spherical” we mean that the particles are spherical or approximately spherical. In other words, as the skilled person would appreciate, in this aspect the particles may not all be true spheres.

The monodisperse particles suitable for use in the colourant compositions of the present invention may be made from any suitable material, including organic and inorganic materials. For example, suitable organic materials include organic polymer particles such as latex, polystyrene, poly(vinyl acetate), polyacrylonitrile, poly(styrene-co-butadiene), polyester, polyamides, polyurethane, poly(methylmethacrylate) and poly(fluoromethylmethacrylate) particles, hydrogel colloids and copolymerisate thereof: poly (N-isopropylacrylamide), poly(acrylic acid), poly(acrylic acid)lhydroxypropylcellulose, dextranlpoly(N-isopropylacrylamide), and dextranlhydroxylpropylcellulose. Also copolymers comprising monomer units of the polymers mentioned before can be used for the invention as well.

Suitable inorganic materials include metal chalcogenide, metal pnictide, silica, metal and metal oxide particles. Examples of suitable metal oxides include, for example, Al₂O₃, TiO₂, SnO₂, Sb₂O₅, Fe₂O₃, ZrO₂, CeO₂ and Y₂O₃. Examples of suitable metals include, for example, gold, copper and silver.

By the term “metal chalcogenide” we mean metal compounds formed with anions from group 16 of the Periodic Table of Elements (according to established IUPAC nomenclature), i.e. oxygen, sulphur, selenium, tellurium and polonium.

By the term “metal pnictide” we mean metal compounds formed with anions from group 15 of the Periodic Table of Elements (according to established IUPAC nomenclature), i.e. nitrogen, phosphorus, arsenic, antimony and bismuth.

In one aspect of the invention, the monodisperse particles comprise organic polymer particles, such as those listed above. In particular, the monodisperse particles may comprise organic polymers selected from polystyrene and poly(methylmethacrylate). Such organic polymer particles are advantageous because they are easy to prepare and can easily be doped with the broad spectrum absorber contrast agent and/or at least one precursor of a broad spectrum absorber contrast agent (for example by surfactant free emulsion polymerisation), i.e. so as to encapsulate the contrast agent. This enables effective control of the concentration of the contrast agent.

Monodisperse particles are commercially available or can be prepared by methods known in the art.

Monodisperse particles made from organic polymer particles may be prepared as dispersions using emulsion, dispersion or suspension polymerisation.

For example, U.S. Pat. No. 6,800,709 describes the preparation of monodisperse particles with a narrow size distribution by free radical polymerization or copolymerization of hydrophobic monomers in a water-based system in the presence of cyclodextrin. Suitable hydrophobic monomers include styrenics, acrylonitrile, methacrylonitrile, acrylates, methacrylates, methacryl amides, acrylamides, maleimides, vinyl ethers, vinyl esters, monoalkylmaleates, dialkyl maleates, fluorinated acrylates and fluorinated methacrylates.

Monodispersed poly(methylmethacrylate) composites may be prepared following the process described by M. Egen, R. Zentel (Macromol. Chem. Phys. 2004, 205, 1479-1488) or are commercially available from Duke Scientific Corporation.

When the broad spectrum absorber contrast agent and/or at least one precursor of a broad spectrum absorber contrast agent is encapsulated in the monodisperse particles, the monodisperse particles typically are formed in the presence of the broad spectrum absorber contrast agent and/or at least one precursor of a broad spectrum absorber contrast agent. For example, monodisperse organic polymer particles (such as polystyrene or poly(methylmethacrylate) particles) that encapsulate at least one broad spectrum absorber contrast agent (such as a dye) and/or at least one precursor of a broad spectrum absorber contrast agent may be prepared using surfactant free emulsion polymerisation, as discussed in more detail below.

Monodisperse particles made from inorganic materials, such as silica particles, may be prepared as dispersions using sol-gel processes.

For example, monodisperse silica spheres can be prepared following the well-known process by Stöber, Fink and Bohn (J. Colloid Interface Sci. 1968, 26,62). The process was later refined by Bogush et al. (J. Non-Crys. Solids 1988, 104, 95). Alternatively, silica particles can be purchased from Blue Helix, Limited or they can be freshly prepared by the process described in U.S. Pat. No. 4,775,520 and U.S. Pat. No. 4,911,903.

Monodisperse silica spheres may also be produced by hydrolytic polycondensation of tetraalkoxysilanes in an aqueous-ammoniacal medium, a sol of primary particles being produced first of all and then the silica particles obtained being brought to the desired particle size by continuous, controlled addition of tetraalkoxysilane. With this process it is possible to produce monodisperse silica spheres having average particle diameters of between 0.05 and 10 μm with a standard deviation of less than 7%.

The surface of the monodisperse particles may be modified so as to implement a variety of effects. The surface groups on the monodisperse particles may be modified and/or changed by performing a chemical reaction. For example, the monodisperse particles may be modified so as to carry charged functional groups, such as carboxylate, sulfate or amine groups. Such modifications would be well known to a person skilled in the art.

By the term “broad spectrum absorber contrast agent” we mean a compound that
(a) absorbs substantially all light having a wavelength in a range that corresponds to the wavelength of visible light, and
(b) eliminates diffuse light.

In the light of the present invention the term “broad spectrum absorber contrast agent” also comprises a mixture of compounds wherein that mixture has the same absorption property as a single broad spectrum absorber contrast agent. Such a mixture still must eliminate diffuse light. That means that each single compound of such a mixture only absorbs the light in a well defined area and only the combination of these compounds provide a broad spectrum absorber.

Therefore a further embodiment of the present invention also relates monodisperse particles capable of forming a colloidal crystal, wherein a broad spectrum absorber contrast agent, which is a mixture of compounds and wherein that mixture has the same absorption property as single broad spectrum absorber contrast agent and wherein the mixture is encapsulated in the particles.

In the following the term “broad spectrum absorber contrast agent” always stands for either a single compound or a mixture of compounds.

It is also possible to encapsulate a precursor material of a broad spectrum absorber contrast agent. That precursor is converted into the broad spectrum absorber contrast agent by using a form of energy (such as light (UV), heat, etc). This conversion usually happens after the encapsulation process.

Suitable precursors for the present invention are metal salts, preferably hydrophilic metal salts, such as nitrates or halogenides. Preferred halogenides are F, Cl or I, whereas Cl is the most preferred halogenide.

The metals are for example alkaline metals, alkaline earth metals, noble metals, rare earth metals or transition metals. Suitable metals are for example K, Ca, Sr, Ba, Zn, Pb, Fe, Ni, Ce, Co, Cr, Cu, Mn, Sn, Al, Ag, Mg, Au, and Cd. Preferred are Ca. Mg, Al, Ag and Zn. Very suitable metal salts are Ag nitrate, Ag halogenide, Fe nitrate and Fe halogenide (especially FeCl₂ and FeCl₃).

As an example AgNO₃ is converted into colloidal silver. It is also possible to use more than one precursor.

As a further example FeCl₂ as well as FeCl₃ are converted into Fe₃O₄ and/or iron hydroxide. Additionally it is also possible to use one or more precursor and another compound which form then a mixture, which has the required broad spectrum absorber contrast agent property. In the following the term “precursor of a broad spectrum absorber contrast agent” always stands for either a single compound or a mixture of compounds.

By this we mean that, as a pure material, the broad spectrum absorber contrast agent absorbs sufficient light so as to appear black or darkly coloured (for example dark blue or dark purple) to the human eye. For example, a broad spectrum absorber contrast agent absorbs most (particularly all) light having a wavelength in a range of from 380 to 780 nm. More specifically, the broad spectrum absorber contrast agent absorbs at least 90% (preferably at least 95%, more preferably 100%) of light having a wavelength in a range of from 380 to 780 nm.

The term “broad spectrum absorber contrast agent” is not intended to encompass those agents that do not absorb substantially all light having a wavelength in a range that corresponds to the wavelength of visible light (and, therefore, that, as a pure material, do not appear black or darkly coloured to the human eye). This term also is not intended to encompass single fluorescent agents, such as single fluorescent dyes or single pigments. But it is possible to encapsulate for example a mixture of various pigments, which have different absorption maxima and wherein the addition of these maxima has a broad spectrum absorber property.

By the term “organic broad spectrum absorber contrast agent” we mean a contrast agent that contains atoms selected from carbon, hydrogen, oxygen, nitrogen and/or sulfur only. By the term “inorganic broad spectrum absorber contrast agent” we mean a contrast agent that contains metal atoms.

In the compositions of the present invention, the broad spectrum absorber contrast agent typically absorbs substantially all of the light that is diffused by the colloidal crystal and that has a wavelength in a range that corresponds to the wavelength of visible light. Contrast agents that are not broad spectrum absorbers as defined herein do not absorb substantially all of the diffused light. The unabsorbed, diffused light dilutes the structural colour effect caused by the direct reflection and/or diffraction of visible light by the colloidal crystal.

The broad spectrum absorber contrast agent may, for example, be a dye or a pigment or a mixture of dyes or a mixture of pigments and dyes as well as a mixture of pigments and dyes, which fulfils the requirements for the broad spectrum absorber contrast agent as defined in the present patent application. A “dye” generally has an affinity to the substrate to which it is applied and generally is in the form of a solution or oil. A “pigment” generally does not have an affinity to the substrate to which it is applied and is in the form of a solid. As the skilled person would appreciate, the exact physical form of the contrast agent is not essential to the present invention and the physical form may change upon application of the colourant composition to a suitable substrate.

The broad spectrum absorber contrast agent is encapsulated in the monodisperse particles. The broad spectrum absorber contrast agent typically is encapsulated in the monodisperse particles before the colloidal crystals are formed.

The broad spectrum absorber contrast agent may be encapsulated in the monodisperse particles in any suitable way. For example, discrete particles of the broad spectrum absorber contrast agent may be embedded inside the monodisperse particles. References herein to the encapsulation of the contrast agent in the monodisperse particles are intended to refer to encapsulation by only one or by two or more of the aforementioned ways of encapsulation.

When the broad spectrum absorber contrast agent is encapsulated in the monodisperse particles, then the colour produced is long lasting. Additionally, the encapsulation of the contrast agent provides environmental advantages, for example because any toxic or undesirable broad spectrum absorber contrast agent material is enclosed in the monodisperse particles and, therefore, is not released into the environment upon application of the colourant composition to a substrate.

Methods for encapsulating a broad spectrum absorber contrast agent or a mixture of compounds with the same property as a broad spectrum absorber contrast agent into monodisperse particles are known and any such suitable method may be used to prepare the colourant compositions of the present invention.

A broad spectrum absorber contrast agent may be encapsulated into monodisperse particles using Surfactant Free Emulsion Polymerisation (SFEP) processes, in which the polymerisation is conducted in the presence of an appropriate contrast agent. For example, Zentel et al. (Chemistry of Materials, 12 (8): 2508) describes a process in which monodisperse organic polymer particles are produced in a Surfactant Free Emulsion Polymerisation (SFEP) in the presence of a water soluble dye. The SFEP process typically produces substantially spherical polymer particles of a narrow size distribution and having surface charges that produce electrostatic repulsion so as to prevent aggregation. The SFEP process also is typically conducted in the absence of emulsifiers which, if present, could bond the particles together so as to make the formation of the colloidal crystals (for example by sedimentation or self-assembly methods) difficult.

A water-insoluble broad spectrum absorber contrast agent may be encapsulated into the monodisperse particles by emulsifying the monodisperse particles in a suitable oil. In this case, the monodisperse particles may additionally encapsulate an oil.

As the person skilled in this art would appreciate, the monodisperse particles may comprise any suitable broad spectrum absorber contrast agent. In one aspect of the invention, the monodisperse particles comprise a broad spectrum absorber contrast agent that is organic.

Another aspect of the present invention relates to
(i) monodisperse particles capable of forming a colloidal crystal as defined above and
(ia) at least one broad spectrum absorber contrast agent and/or at least one precursor of a broad spectrum absorber contrast agent, which is encapsulated in the monodisperse particles.

In another aspect of the invention, the monodisperse particles comprise a broad spectrum absorber contrast agent that is inorganic.

Suitable broad spectrum absorber contrast agents include absorbing elements such as silver, dyes such as Alizarin Blue Black and Brilliant Blue Black, as well as pigments such as carbon black (for example the carbon black product line from Degussa, such as Purex® LS 35 and Corax® N 115), iron hydroxide and iron oxide black.

Another aspect of the present invention relates to monodisperse particles comprising
(i) monodisperse particles capable of forming a colloidal crystal as defined above and
(ia) at least one broad spectrum absorber contrast agents chosen from the group consisting of Alizarin Blue Black and Brilliant Blue Black, as well as pigments such as carbon black (for example the carbon black product line from Degussa, such as Purex® LS 35 and Corax® N 115), iron hydroxide and iron oxide black, which is encapsulated in the monodisperse particles.

The monodisperse particles of the present invention are capable of forming a colloidal crystal, for example upon application of the colourant composition to a substrate. In another related aspect of the present invention, the colourant compositions can comprise colloidal crystals formed from monodisperse particles. Typically, the colloidal crystals reflect and/or diffract light having a wavelength in a range that corresponds to the wavelength of visible light. There typically is also some diffusion of the light by the colloidal crystal.

For the avoidance of doubt, the wavelength of visible light is, for example, in the range of from 380 to 780 nm. Thus, the colloidal crystals appear coloured to the human eye.

For the avoidance of doubt, references herein to “a colloidal crystal” are intended to relate to one or more colloidal crystals.

By the term “colloidal crystal” we mean a regular array of monodisperse particles having a substantially regular or constant spacing there between. Thus, the array of monodisperse particles forms a dispersed phase arranged in a continuous phase (or matrix). The continuous phase (or matrix) may comprise a gas, a liquid or a solid of a different refractive index to the dispersed phase.

As the skilled person would appreciate, a colloidal crystal may, however, contain some impurities and/or defects. The levels of impurities and/or defects typically will depend on the materials and methods of preparation used.

The term “colloidal crystal” has the same meaning as the term “super-lattice”. A colloidal crystal or super-lattice is a type of photonic crystal, which is an optical, artificial structure characterised by 2D or 3D periodic arrangements of dielectric material which lead to the formation of energy band structures for electromagnetic waves propagating in them.

As discussed above, the colloidal crystals appear coloured to the human eye. In other words, the colloidal crystals reflect and/or diffract light in the visible spectrum.

The crystal colour or colours observed by the human eye depend principally on two factors. These factors are the lattice spacing within the colloidal crystal and the refractive index of the disperse and continuous phases. Both of these factors affect the wavelength of light reflected and/or diffracted by the colloidal crystal.

The lattice spacing is determined by factors such as the size of the monodisperse particles. For example, monodisperse particles having a rms diameter in the range of from 250 to 510 nm can be used to form colloidal crystals that have colours ranging from blue and red to green and yellow. Colloidal crystals can have different colours when viewed from different angles because the lattice spacing can be different in different axes of the crystal. Provided that the lattice spacing in at least one axis results in the reflection and/or diffraction of light with a wavelength in the visible spectrum then the colloidal crystal will appear to the human eye to be coloured.

The colloidal crystal (once formed) may have a lattice spacing in at least one axis in a range that corresponds to the wavelength of visible light. Preferably, the lattice spacing in at least one axis is in a range of from 380 to 700 nm.

References above to the refractive index of the disperse and continuous phases are especially intended to relate to the difference in refractive index between the disperse and continuous phases. This is known as the “refractive index contrast”, which is the ratio of the refractive index of the two phases.

Methods of forming colloidal crystals from monodisperse particles are known in the art. For example, sedimentation or self-assembly methods may be used.

The sedimentation method of forming colloidal crystals comprises the steps of placing a solution or suspension of monodisperse particles in a suitable carrier or solvent in an appropriate container or vessel and then simply allowing the monodisperse particles to form the colloidal crystals as they settle in the container or vessel.

The sedimentation process is driven by gravitational forces. The gravitational force counteracts the Brownian motion of the monodisperse particles in the dispersion. Above a critical volume content (for example greater than about 50% by volume), an equilibrium-state between a disorganised liquid phase and a denser colloidal crystalline phase is developed. This process is, however, typically very slow. For example, polystyrene particles with a rms diameter of 1 μm take approximately one month to reach the aforementioned equilibrium state. This period of time can be reduced by using centrifugal forces, for example by using a centrifuge.

Thus, the monodisperse particles may be formed into colloidal crystals by placing the solution or suspension of monodisperse particles in a suitable carrier or solvent in an appropriate container or vessel and the centrifuging the solution or suspension. The carrier or solvent may then be removed from the colloidal crystals that are formed by any suitable method. For example, the carrier or solvent may be removed by evaporation or by decanting the carrier or solvent. Once the carrier or solvent has been removed, the colloidal crystals may be analysed by any suitable method, such as by Transmission Electron Microscopy and Scanning Electron Microscopy methods.

Typically, the sedimentation process provides colloidal crystals of a face-centred-cubic structure. However, as the skilled person would appreciate, some defects are unavoidable, such as the formation of some colloidal crystals of a body-centred-cubic structure.

The self-assembly method of forming colloidal crystals comprises the step of providing a suspension or solution of monodisperse particles in a suitable carrier or solvent and contacting a suitable substrate with the suspension or solution, for example by placing the suitable substrate in the suspension or solution. Slow evaporation of the carrier or solvent under the appropriate conditions leaves a deposit of colloidal crystals on the substrate. Typically, a carrier or solvent (such as an alcohol) is selected which will evaporate within a convenient time scale.

The self-assembly method is driven by capillary forces. This method provides a template form, referred to in the art as a “planar opal”. The capillary forces act to uniformly deposit a specific number of layers (for example 25) of close-packed monodisperse particles onto the substrate. This method was used by Denkov et al. to make two-dimensionally periodic monolayers of monodisperse particles (see “Two-Dimensional Crystallization”, Nature, Vol. 361, p. 26 (1993) and U.S. Pat. No. 5,540,951) and was extended by Jiang et al. to make three-dimensional opaline structures (see “Template-Directed Preparation of Macroporous Polymers with Oriented and Crystalline Arrays of Voids”, Journal of the American Chemical Society, Vol. 121, pp. 11630-11637 (1999)). In the method of Jiang et al., a glass substrate was placed vertically in a solution of monodisperse particles. Slow evaporation of the solvent under the appropriate conditions leaves a deposit of three-dimensionally ordered particles in a face-centred cubic lattice, i.e. colloidal crystals.

In the self-assembly method, the monodisperse particles are deposited directly onto the substrate. Thus, the substrate may be the substrate that is to be coloured using the colourant compositions of the present invention.

Whilst the self-assembly method typically forms colloidal crystals in which point defects may remain, the crystals formed by this method have the potential of being single crystals. Thus, the colloidal crystals formed by the self-assembly method are often superior to those formed by the sedimentation method because they are not polycrystalline, they are of a well-defined thickness, and they have a known crystal orientation.

Apparatus for forming colloidal crystals by the self-assembly method are known. For example, Nagayama et al. (J. Phys.: Condens. Matter; 1994 (6), A395) describes a mechanical apparatus, which acts to pull a glass slide out of a highly diluted dispersion of monodisperse particles. The monodisperse particles are pulled onto the glass slide and ordered so as to form colloidal crystals. This ordering of the monodisperse particles occurs because the surface of the liquid on the glass slide obtains a curvature through dewetting. Thus, a thin liquid film is observed on the glass slide, which has a decreasing thickness towards the edges. In the position where the film is thinner than the diameter of the colloidal crystals, the monodisperse particles are pulled out together, due to capillary forces, into a dense packet. During evaporation of the solvent, a convection effect is created in the liquid, through which further monodisperse particles are transported along. The glass slide is pulled out of the dispersion at a speed that corresponds to the growth rate of the colloidal crystals. The crystalline order is consequently gained by capillary forces. The direction of the crystal growth is determined by the movement of the glass slide, which counteracts to the effective diffuse mobility of the particles. Very thin layers of colloidal crystal are generated, for example packed in from one to three hexagonal sorted layers.

This procedure was further developed by Colvin et al. (Phys. Rev. B; 2001 (64), 205103) using dispersions of silica particles in ethanol. The mechanical extraction of the glass slide was replaced by a slow evaporation process of the solvent from a sample holder, which was positioned vertically in a narrow vial. The colloidal crystals grow continuously on the glass surface of the sample holder, as the liquid level decreases by evaporation. The samples are allowed to stand for a few hours. This method forms layers of colloidal crystals of controlled thickness, as observed by Scanning Electron Microscopy methods.

According to an aspect of the present invention, there is provided a colloidal crystal formed from monodisperse particles and that reflects and/or diffracts light having a wavelength in a range that corresponds to the wavelength of visible light, wherein the colloidal crystal comprises at least one broad spectrum absorber contrast agent and/or at least one precursor of a broad spectrum absorber contrast agent, which is encapsulated in the monodisperse particles. According to another related aspect of the present invention, there is provided a colloidal crystal formed from monodisperse particles and that reflects and/or diffracts light having a wavelength in a range that corresponds to the wavelength of visible light, wherein the colloidal crystal comprises at least broad spectrum absorber contrast agent and/or at least one precursor of a broad spectrum absorber contrast agent which is encapsulated in the monodisperse particles. The broad spectrum absorber contrast agent and/or at least one precursor of a broad spectrum absorber contrast agent is encapsulated in the monodisperse particles and may additionally be positioned in interstices between the monodisperse particles and/or may be additionally located on the surface of the monodisperse particles. The colloidal crystal may be formed by any suitable method, for example by a method as discussed above.

Therefore the present invention also relates to
a colloidal crystal formed from monodisperse particles as defined above.

The monodisperse particles, wherein at least one broad spectrum absorber contrast agent and/or at least one precursor of a broad spectrum absorber contrast agent is encapsulated as described above as well as the colloidal crystal as described above can be used in a colourant composition.

Therefore a further embodiment of the present invention relates to a colourant composition (CC 1) comprising monodisperse particles as described above and/or a colloidal crystal as described above.

All the preferences for the monodisperse particles (including the broad spectrum absorber contrast agent and the precursor of a broad spectrum absorber contrast agent) are (of course) also applicable when the monodisperse particles and/or the colloidal crystals are incorporated into a colourant composition.

It is also possible to combine the monodisperse particles wherein at least one broad spectrum absorber contrast agent and/or at least one precursor of a broad spectrum absorber contrast agent is encapsulated with monodisperse particles wherein no broad spectrum absorber contrast agent and no precursor of a broad spectrum absorber contrast agent is encapsulated.

In other words, this is a mixture of encapsulated monodisperse particles with encapsulated broad spectrum absorber contrast agent and/or at least one precursor of a broad spectrum absorber contrast agent and monodisperse particles, which comprise no encapsulated broad spectrum absorber contrast agent and no precursor of a broad spectrum absorber contrast agent.

A further embodiment of the present invention relates to a colourant composition (CC 2) comprising
(i) monodisperse particles as described above wherein at least broad spectrum absorber contrast agent and/or at least one precursor of a broad spectrum absorber contrast agent and
(ia) monodisperse particles, wherein no broad spectrum absorber contrast agent and no precursor of a broad spectrum absorber contrast agent is encapsulated.

It is clear that in such a colourant composition which comprises a mixture of monodisperse particles which encapsulates at least one broad spectrum absorber contrast agent and/or at least one precursor of a broad spectrum absorber contrast agent and which do not comprise a broad spectrum absorber contrast agent and/or a precursor of a broad spectrum absorber contrast agent, the monodisperse particles are the same monodisperse particles. At least they must have the same dimensions (not essentially consists of the same material).

In addition to the encapsulated broad spectrum absorber contrast agent and/or at least one precursor of a broad spectrum absorber contrast agent it is also possible to use at least one non-encapsulated broad spectrum absorber contrast agent and/or at least one precursor of a broad spectrum absorber contrast agent.

Therefore a further embodiment of the present invention is a colourant composition which additionally comprises at least one broad spectrum absorber contrast agent and/or at least one precursor of a broad spectrum absorber contrast agent, which are not encapsulated by the monodisperse particles.

Of course it is also possible to have a colourant composition which comprises
(a) monodisperse particles which comprise at least one encapsulated broad spectrum absorber contrast agent and/or at least one precursor of a broad spectrum absorber contrast agent, and
(b) monodisperse particles wherein no broad spectrum absorber contrast agent and no precursor of a broad spectrum absorber contrast agent is encapsulated, and
(c) at least one broad spectrum absorber contrast agent and/or at least one precursor of a broad spectrum absorber contrast agent which is not encapsulated.

The amount of such non-encapsulated broad spectrum absorber contrast agent and/or at least one precursor of a broad spectrum absorber contrast agent in the colourant composition can be up to 3 wt-%, based on the total weight of the colourant composition. Preferably it is 0.0001-3 wt-%, more preferably 0.0001-1 wt-%, especially preferably 0.0001-0.5 wt-%, based on the total weight of the colourant composition.

In another aspect of the present invention, the broad spectrum absorber contrast agent and/or at least one precursor of a broad spectrum absorber contrast agent, which is not encapsulated, may be positioned in interstices between the encapsulated monodisperse particles in the colloidal crystal. In this case, the broad spectrum absorber contrast agent and/or at least one precursor of a broad spectrum absorber contrast agent typically is positioned in the interstices as the colloidal crystals are formed, for example as the colourant composition is applied to a substrate. This may be achieved by forming the colloidal crystals in the presence of the broad spectrum absorber contrast agent and/or at least one precursor of a broad spectrum absorber contrast agent so that the broad spectrum absorber contrast agent and/or at least one precursor of a broad spectrum absorber contrast agent occupies the interstices as the colloidal crystals form. Alternatively, a solvent may be evaporated from a colourant composition that comprises a colloidal crystal and a broad spectrum absorber contrast agent (such as a dye) and/or at least one precursor of a broad spectrum absorber contrast agent in a suitable solvent, so as to leave the broad spectrum absorber contrast agent occupying the interstices in the colloidal crystal. Alternatively, the broad spectrum absorber contrast agent and/or at least one precursor of a broad spectrum absorber contrast agent may simply be deposited in the interstices in a colloidal crystal.

As the skilled person would appreciate, although it is possible that 100% of the interstices within the colloidal crystals may be occupied by a broad spectrum absorber contrast agent and/or at least one precursor of a broad spectrum absorber contrast agent, this is not necessary. In other words, the interstices within the colloidal particles may be wholly or partially occupied by the broad spectrum absorber contrast agent and/or at least one precursor of a broad spectrum absorber contrast agent.

In another aspect of the present invention, the broad spectrum absorber contrast agent and/or at least one precursor of a broad spectrum absorber contrast agent may additionally be located on the surface of the encapsulated monodisperse particles, i.e. so as to form a coating on the particles. The broad spectrum absorber contrast agent and/or at least one precursor of a broad spectrum absorber contrast agent may, for example, be adsorbed onto the surface of the encapsulated monodisperse particles by physical interactions (such as by hydrophilic, hydrophobic or electrostatic interactions or by hydrogen bonding) or by chemical bonding (such as covalent chemical bonding). Whilst it is possible that 100% of the surface area of the monodisperse particles is coated with the broad spectrum absorber contrast agent and/or at least one precursor of a broad spectrum absorber contrast agent, this is not essential. For example, it is sufficient that at least 90% of the surface area of the encapsulated monodisperse particles be coated with the broad spectrum absorber contrast agent and/or at least one precursor of a broad spectrum absorber contrast agent.

The broad spectrum absorber contrast agent and/or at least one precursor of a broad spectrum absorber contrast agent may be located in the colloidal crystals of the present invention in only one of or in two or more of the locations discussed above. For example, in one aspect of the present invention, the broad spectrum absorber contrast agent and/or at least one precursor of a broad spectrum absorber contrast agent is encapsulated in the monodisperse particles only. In another aspect of the present invention, the broad spectrum absorber contrast agent and/or at least one precursor of a broad spectrum absorber contrast agent is both encapsulated in the monodisperse particles and positioned in interstices between the monodisperse particles in the colloidal crystal. Other combinations of broad spectrum absorber contrast agent and/or at least one precursor of a broad spectrum absorber contrast agent locations are, of course, also possible.

The broad spectrum absorber contrast agent and/or at least one precursor of a broad spectrum absorber contrast agent which is not encapsulated can be chosen from the same types of agents and precursors as defined for the encapsulated embodiment.

The colourant compositions of the present invention may typically be in liquid form; semi-liquid form including lotions, pastes, creams, mousse, waxes; or solid form including powders for example laundry powders or tablets.

The colourant composition according to the present invention comprise from 0.01 wt-% to 70 wt-%, based on the total weight of the colouring composition monodisperse particles wherein at least one broad spectrum absorber contrast agent and/or at least one precursor of a broad spectrum absorber contrast agent is encapsulated.

A further embodiment of the present invention relates to a colourant composition (CC 3) comprising
0.01 wt-% to 70 wt-%, based on the total weight of the colourant composition, of encapsulated monodisperse particles comprising
   (i) 97-99.999 wt-%, preferably 99-99.9999 wt-%, more preferably 99.5-99.9999 wt-%, based on the total weight of the monodisperse particles, of the monodisperse particle material and
   (ii) 0.0001-3 wt-%, preferably 0.0001-1 wt-%, more preferably 0.0001-0.5 wt-%, based on the total weight of the monodisperse particles, of at least one broad spectrum absorber contrast agent and/or at least one precursor of a broad spectrum absorber contrast agent.

A further embodiment of the present invention relates to a colourant composition (CC 3′) comprising
0.01 wt-% to 70 wt-%, based on the total weight of the colourant composition, of encapsulated monodisperse particles comprising
   (i) 97-99.999 wt-%, preferably 99-99.9999 wt-%, more preferably 99.5-99.9999 wt-%, based on the total weight of the monodisperse particles, of the monodisperse particle material and
   (ii) 0.0001-3 wt-%, preferably 0.0001-1 wt-%, more preferably 0.0001-0.5 wt-%, based on the total weight of the monodisperse particles, of at least one broad spectrum absorber contrast agent and/or at least one precursor of a broad spectrum absorber contrast agent, with the proviso that the encapsulated monodisperse particle are not these of example 1 of PCT Application No PCT/EP2006/010882.

A further embodiment of the present invention relates to a colourant composition (CC 3″) comprising
0.01 wt-% to 70 wt-%, based on the total weight of the colourant composition, of encapsulated monodisperse particles comprising
   (i) 97-99.999 wt-%, preferably 99-99.9999 wt-%, more preferably 99.5-99.9999 wt-%, based on the total weight of the monodisperse particles, of the monodisperse particle material and
   (ii) 0.0001-3 wt-%, preferably 0.0001-1 wt-%, more preferably 0.0001-0.5 wt-%, based on the total weight of the monodisperse particles, of at least one broad spectrum absorber contrast agent and/or at least one precursor of a broad spectrum absorber contrast agent, with the proviso that the encapsulated monodisperse particles are not consisting of methylmethacrylate and Alizarin Blue Black.

When the colourant composition is in the form of a liquid, gel, mousse, wax or paste, then the composition comprises at least one solvent.

A further embodiment of the present invention relates to a colourant composition (CC 4) comprising
(i) monodisperse particles, wherein at least one broad spectrum absorber contrast agent and/or at least one precursor of a broad spectrum absorber contrast agent is encapsulated, and
(ii) at least one solvent.

Preferably the solvent is an organic solvent, which can be polar or nonpolar. Examples of polar solvents include water, alcohols (mono or poly), esters, ketones and ethers, particularly mono- and di-alkyl ethers of glycols and polyglycols such as monomethyl ethers of mono-, di- and tri-propylene glycols and the mono-n-butyl ethers of ethylene, diethylene and triethylene glycols.

Examples of nonpolar solvents include aliphatic and aromatic hydrocarbons having at least six carbon atoms and mixtures thereof including refinery distillation products and by-products.

The colourant composition can be prepared as an aqueous or as a nonaqueous solution.

Therefore, another embodiment of the present invention relates to a colourant composition as described above, wherein the formulation is nonaqueous.

Therefore, another embodiment of the present invention relates to a colourant composition as described above, wherein the formulation is aqueous.

A further embodiment of the present invention relates to a colourant composition (CC 5) comprising
(i) monodisperse particles as defined above capable of forming a colloidal crystal that diffracts light having a wavelength in a range that corresponds to the wavelength of visible light, wherein at least one broad spectrum absorber contrast agent and/or at least one precursor of a broad spectrum absorber contrast agent, is encapsulated in the monodisperse particles,
(iia) water, and
(iib) optionally at least one solvent, chosen from the group consisting of alcohols, esters, ketones, ethers and aliphatic and aromatic hydrocarbons having at least six carbon atoms and mixtures thereof including refinery distillation products and by-products.

A further embodiment of the present invention relates to a colourant composition (CC 6) comprising
(i) a colloidal crystal formed from monodisperse particles as defined above, wherein at least one broad spectrum absorber contrast agent and/or at least one precursor of a broad spectrum absorber contrast agent is encapsulated in the monodisperse particles,
(iia) water, and
(iib) optionally at least one solvent, chosen from the group consisting of alcohols, esters, ketones, ethers and aliphatic and aromatic hydrocarbons having at least six carbon atoms and mixtures thereof including refinery distillation products and by-products.

Therefore, the present invention also relates to a colourant composition (CC 7) comprising
(i) monodisperse particles as defined above capable of forming a colloidal crystal that diffracts light having a wavelength in a range that corresponds to the wavelength of visible light, wherein at least one broad spectrum absorber contrast agent and/or at least one precursor of a broad spectrum absorber contrast agent, is encapsulated in the monodisperse particles, and
(ii) at least one solvent, chosen from the group consisting of alcohols, esters, ketones, ethers and aliphatic and aromatic hydrocarbons having at least six carbon atoms and mixtures thereof including refinery distillation products and by-products.

A further embodiment of the present invention relates to a colourant composition (CC 8) comprising
(i) a colloidal crystal formed from monodisperse particles as defined above, wherein at least one broad spectrum absorber contrast agent, characterized in that the broad spectrum absorber contrast agent and/or at least one precursor of a broad spectrum absorber contrast agent is encapsulated in the monodisperse particles, and
(ii) at least one solvent, chosen from the group consisting of alcohols, esters, ketones, ethers and aliphatic and aromatic hydrocarbons having at least six carbon atoms and mixtures thereof including refinery distillation products and by-products.

But, even when no water is deliberately added to the nonaqueous composition, some adventitious water may be carried into the composition, but generally this will be no more than about 2 wt-%-4 wt-%, based on the total weight of colourant composition. By definition, the nonaqueous composition of this invention will have no more than about 10 wt-%, and preferably no more than about 5 wt-% water based on the total weight of colourant composition.

The amount of solvent in a colourant composition according to the present invention is typically in the range of about 10 wt-% to about 99.99 wt-%, preferably from about 20 wt-% to about 99.9 wt-%, and more preferably from about 30 wt-% to about 99.9 wt-%, based on total weight of the colourant composition.

The amount of solvent, which is part of the inventive formulation, can very a lot. The reasons for that are the same as explained for the monodisperse particles above.

When the composition is used as a concentrate, which is to be diluted (with water and/or other solvents), then the amount of solvents is low, usually between 30 wt-% and 70 wt-%, based on the total weight of the colourant composition. In certain cases the colouring composition can comprise even less that 30 wt-%.

When the formulation is in a ready-to-use form then the solvent content can be up to 99.5 wt-%, based on the total weight of the colourant composition.

It is obvious that the amount of solvent also depends on the substrate which is to be coated or printed as well as on the hue which needs to be obtained.

Therefore, the present invention also relates to a concentrated colourant composition, wherein the amount of solvent lies between 30 wt-% and 70 wt-%, preferably between 40 wt-% and 70 wt-%, more preferably between 50 wt-% and 70 wt-%, based on the total weight of colourant composition.

The present invention also relates to a colourant composition, wherein the amount of water lies between 70 wt-% and 99.99 wt-%, -%, preferably between 70 wt-% and 99.9 wt-%, more preferably between 80 wt-% and 99.9 wt-%, based on the total weight of the colourant composition.

It also to be stated that the amount of the encapsulated monodisperse particles as well as of the solvent can vary depending of the physical form of the composition, that means the concentration can vary in case the colourant composition is a liquid, gel or a paste.

But as said above the broad spectrum absorber contrast agent and/or at least one precursor of a broad spectrum absorber contrast agent can also additionally be added to the colourant composition and therefore the molar amount of the broad spectrum absorber contrast agent can be larger.

On the other hand it is also possible to add monodisperse particles which do not comprise a broad spectrum absorber contrast agent and/or at least one precursor of a broad spectrum absorber contrast agent.

When the colourant compositions of the present invention are used to colour natural substrates, such as hair, nail, tooth and natural fabrics such as wool or cotton, the compositions typically comprise from 0.01 to 2% (preferably from 0.01 to 1%) by weight of the monodisperse particles or colloidal crystals and from 0.01 to 2% (preferably from 0.01 to 1%) by weight of the broad spectrum absorber contrast agent and/or at least one precursor of a broad spectrum absorber contrast agent. When the colourant compositions of the present invention are used to colour synthetic substrates, the compositions typically comprise from 0.5 to 15% (preferably from 1 to 10%) by weight of the monodisperse particles or colloidal crystals and from 0.5 to 15% (preferably from 1 to 10%) by weight of the broad spectrum absorber contrast agent and/or at least one precursor of a broad spectrum absorber contrast agent.

The present invention also relates to a colourant composition, wherein the amount of the encapsulated monodisperse particles lies between 0.5 wt-% and 30 wt-%, based on the total weight of the colouring composition.

A further embodiment of the present invention relates to a colourant composition (CC 9) comprising
(i) 0.01 wt-% to 70 wt-%, based on the total weight of the colourant composition, of encapsulated monodisperse particles comprising
   (i) 97-99.999 wt-%, preferably 99-99.9999 wt-%, more preferably 99.5-99.9999 wt-%, based on the total weight of the monodisperse particles, of the monodisperse particle material, and
   (ii) 0.0001-3 wt-%, preferably 0.0001-1 wt-%, more preferably 0.0001-0.5 wt-%, based on the total weight of the monodisperse particles, of at least one broad spectrum absorber contrast agent and/or at least one precursor of a broad spectrum absorber contrast agent, which is encapsulated, and
(ii) 30 wt-% and 99.99 wt-%, based on the total weight of the colourant composition, of at least one solvent.

A further embodiment of the present invention relates to a colourant composition (CC 9′) comprising
(i) 0.01 wt-% to 70 wt-%, based on the total weight of the colourant composition, of encapsulated monodisperse particles comprising
   (i) 97-99.999 wt-%, preferably 99-99.9999 wt-%, more preferably 99.5-99.9999 wt-%, based on the total weight of the monodisperse particles, of the monodisperse particle material and
   (ii) 0.0001-3 wt-%, preferably 0.0001-1 wt-%, more preferably 0.0001-0.5 wt-%, based on the total weight of the monodisperse particles, of at least one broad spectrum absorber contrast agent and/or at least one precursor of a broad spectrum absorber contrast agent, with the proviso that the encapsulated monodisperse particles are not these of example 1 of PCT Application No PCT/EP2006/010882, and
(ii) 30 wt-% and 99.99 wt-%, based on the total weight of the colourant composition, of at least one solvent.

A further embodiment of the present invention relates to a colourant composition (CC 9″) comprising
(i) 0.01 wt-% to 70 wt-%, based on the total weight of the colourant composition, of encapsulated monodisperse particles comprising
   (i) 97-99.999 wt-%, preferably 99-99.9999 wt-%, more preferably 99.5-99.9999 wt-%, based on the total weight of the monodisperse particles, of the monodisperse particle material and
   (ii) 0.0001-3 wt-%, preferably 0.0001-1 wt-%, more preferably 0.0001-0.5 wt-%, based on the total weight of the monodisperse particles, of at least one broad spectrum absorber contrast agent and/or at least one precursor of a broad spectrum absorber contrast agent, with the proviso that the encapsulated monodisperse particles are not consisting of methylmethacrylate and Alizarin Blue Black, and
(ii) 30 wt-% and 99.99 wt-%, based on the total weight of the colourant composition, of at least one solvent.

A further embodiment of the present invention relates to a colourant composition (CC 10) comprising
(i) 0.01 wt-% to 70 wt-%, based on the total weight of the colourant composition, of encapsulated monodisperse particles, comprising
   97-99.999 wt-%, preferably 99-99.9999 wt-%, more preferably 99.5-99.9999 wt-%, based on the total weight of the encapsulated monodisperse particles, of the monodisperse particle material chosen from the group consisting of organic polymer particles such as latex, acrylic, polystyrene, poly(vinyl acetate), polyacrylonitrile, poly(styrene-co-butadiene), polyester, polyamides, polyurethane, poly(methylmethacrylate) and poly(fluoromethylmethacrylate) particles; inorganic materials such as metal chalcogenide, metal pnictide, silica, metal and metal oxide particles; metal oxides such as Al₂O₃, TiO₂, SnO₂, Sb₂O₅, Fe₂O₃, ZrO₂, CeO₂ and Y₂O₃; and metals such as gold, copper and silver,
   capable of forming a colloidal crystal that diffracts light having a wavelength in a range that corresponds to the wavelength of visible light, and
   0.0001-3 wt-%, preferably 0.0001-1 wt-%, more preferably 0.0001-0.5 wt-%, based on the total weight of the encapsulated monodisperse particles, of at least one broad spectrum absorber contrast agent and/or at least one precursor of a broad spectrum absorber contrast agent chosen from the group consisting of Alizarin Blue Black, Brilliant Blue Black, carbon black, black iron oxide, iron hydroxide, iron oxide black, silver oxide black, K, Ca, Sr, Ba, Zn, Pb, Fe, Ni, Ce, Co, Cr, Cu, Mn, Sn, Al, Ag, Mg, Au, Cd, Ag nitrate, Ag halogenide, Fe nitrate and Fe halogenide, which is encapsulated,
(iia) 30 wt-% and 99.99 wt-%, based on the total weight of the colourant composition, of water, and
(iib) optionally 0.1 wt-% and 89.99 wt-%, based on the total weight of the colourant composition of at least one solvent, chosen from the group consisting of alcohols, esters, ketones, ethers and aliphatic and aromatic hydrocarbons having at least six carbon atoms and mixtures thereof including refinery distillation products and by-products.

A further embodiment of the present invention relates to a colourant composition (CC 10′) comprising
(i) 0.01 wt-% to 70 wt-%, based on the total weight of the colourant composition, of encapsulated monodisperse particles, comprising
   97-99.999 wt-%, preferably 99-99.9999 wt-%, more preferably 99.5-99.9999 wt-%, based on the total weight of the encapsulated monodisperse particles, of the monodisperse particle material chosen from the group consisting of organic polymer particles such as latex, acrylic, polystyrene, poly(vinyl acetate), polyacrylonitrile, poly(styrene-co-butadiene), polyester, polyamides, polyurethane, poly(methylmethacrylate) and poly(fluoromethylmethacrylate) particles; inorganic materials such as metal chalcogenide, metal pnictide, silica, metal and metal oxide particles; metal oxides such as Al₂O₃, TiO₂, SnO₂, Sb₂O₅, Fe₂O₃, ZrO₂, CeO₂ and Y₂O₃; and metals such as gold, copper and silver,
   capable of forming a colloidal crystal that diffracts light having a wavelength in a range that corresponds to the wavelength of visible light, and 0.0001-3 wt-%, preferably 0.0001-1 wt-%, more preferably 0.0001-0.5 wt-%, based on the total weight of the encapsulated monodisperse particles, of at least one broad spectrum absorber contrast agent and/or at least one precursor of a broad spectrum absorber contrast agent chosen from the group consisting of Alizarin Blue Black, Brilliant Blue Black, carbon black, black iron oxide, iron hydroxide, iron oxide black, silver oxide black, K, Ca, Sr, Ba, Zn, Pb, Fe, Ni, Ce, Co, Cr, Cu, Mn, Sn, Al, Ag, Mg, Au, Cd, Ag nitrate, Ag halogenide, Fe nitrate and Fe halogenide, which is encapsulated, with the proviso that the encapsulated monodisperse particle is not these of example 1 of PCT Application No PCT/EP2006/010882,
(iia) 30 wt-% and 99.99 wt-%, based on the total weight of the colourant composition, of water, and
(iib) optionally 0.1 wt-% and 89.99 wt-%, based on the total weight of the colourant composition of at least one solvent, chosen from the group consisting of alcohols, esters, ketones, ethers and aliphatic and aromatic hydrocarbons having at least six carbon atoms and mixtures thereof including refinery distillation products and by-products.

A further embodiment of the present invention relates to a colourant composition (CC 10″) comprising
(i) 0.01 wt-% to 70 wt-%, based on the total weight of the colourant composition, of encapsulated monodisperse particles, comprising
   97-99.999 wt-%, preferably 99-99.9999 wt-%, more preferably 99.5-99.9999 wt-%, based on the total weight of the encapsulated monodisperse particles, of the monodisperse particle material chosen from the group consisting of organic polymer particles such as latex, acrylic, polystyrene, poly(vinyl acetate), polyacrylonitrile, poly(styrene-co-butadiene), polyester, polyamides, polyurethane, poly(methylmethacrylate) and poly(fluoromethylmethacrylate) particles; inorganic materials such as metal chalcogenide, metal pnictide, silica, metal and metal oxide particles; metal oxides such as Al₂O₃, TiO₂, SnO₂, Sb₂O₅, Fe₂O₃, ZrO₂, CeO₂ and Y₂O₃; and metals such as gold, copper and silver,
   capable of forming a colloidal crystal that diffracts light having a wavelength in a range that corresponds to the wavelength of visible light, and 0.0001-3 wt-%, preferably 0.0001-1 wt-%, more preferably 0.0001-0.5 wt-%, based on the total weight of the encapsulated monodisperse particles, of at least one broad spectrum absorber contrast agent and/or at least one precursor of a broad spectrum absorber contrast agent chosen from the group consisting of Alizarin Blue Black, Brilliant Blue Black, carbon black, black iron oxide, iron hydroxide, iron oxide black, silver oxide black, K, Ca, Sr, Ba, Zn, Pb, Fe, Ni, Ce, Co, Cr, Cu, Mn, Sn, Al, Ag, Mg, Au, Cd, Ag nitrate, Ag halogenide, Fe nitrate and Fe halogenide, which is encapsulated, with the proviso that the encapsulated monodisperse particles are not consisting of methylmethacrylate and Alizarin Blue Black,
(iia) 30 wt-% and 99.99 wt-%, based on the total weight of the colourant composition, of water, and
(iib) optionally 0.1 wt-% and 89.99 wt-%, based on the total weight of the colourant composition of at least one solvent, chosen from the group consisting of alcohols, esters, ketones, ethers and aliphatic and aromatic hydrocarbons having at least six carbon atoms and mixtures thereof including refinery distillation products and by-products.

A further embodiment of the present invention relates to a colourant composition (CC 11) comprising
(i) 0.01 wt-% to 70 wt-%, based on the total weight of the colourant composition, of encapsulated monodisperse particles, comprising
   97-99.999 wt-%, preferably 99-99.9999 wt-%, more preferably 99.5-99.9999 wt-%, based on the total weight of the encapsulated monodisperse particles, of the monodisperse particles chosen from the group consisting of organic polymer particles such as latex, acrylic, polystyrene, poly(vinyl acetate), polyacrylonitrile, poly(styrene-co-butadiene), polyester, polyamides, polyurethane, poly(methylmethacrylate) and poly(fluoromethylmethacrylate) particles; inorganic materials such as metal chalcogenide, metal pnictide, silica, metal and metal oxide particles; metal oxides such as Al₂O₃, TiO₂, SnO₂, Sb₂O₅, Fe₂O₃, ZrO₂, CeO₂ and Y₂O₃; and metals such as gold, copper and silver,
   capable of forming a colloidal crystal that diffracts light having a wavelength in a range that corresponds to the wavelength of visible light, and 0.0001-3 wt-%, preferably 0.0001-1 wt-%, more preferably 0.0001-0.5 wt-%, based on the total weight of the encapsulated monodisperse particles, of at least one broad spectrum absorber contrast agent and/or at least one precursor of a broad spectrum absorber contrast agent chosen from the group consisting of Alizarin Blue Black, Brilliant Blue Black, carbon black, black iron oxide, iron hydroxide, iron oxide black, silver oxide black, K, Ca, Sr, Ba, Zn, Pb, Fe, Ni, Ce, Co, Cr, Cu, Mn, Sn, Al, Ag, Mg, Au, Cd, Ag nitrate, Ag halogenide, Fe nitrate and Fe halogenide, which is encapsulated,
(ii) 30 wt-% and 99.99 wt-%, based on the total weight of the colourant composition, of at least one solvent, chosen from the group consisting of alcohols, esters, ketones, ethers and aliphatic and aromatic hydrocarbons having at least six carbon atoms and mixtures thereof including refinery distillation products and by-products.

A further embodiment of the present invention relates to a colourant composition (CC 11′) comprising
(i) 0.01 wt-% to 70 wt-%, based on the total weight of the colourant composition, of encapsulated monodisperse particles, comprising
   97-99.999 wt-%, preferably 99-99.9999 wt-%, more preferably 99.5-99.9999 wt-%, based on the total weight of the encapsulated monodisperse particles, of the monodisperse particle material chosen from the group consisting of organic polymer particles such as latex, acrylic, polystyrene, poly(vinyl acetate), polyacrylonitrile, poly(styrene-co-butadiene), polyester, polyamides, polyurethane, poly(methylmethacrylate) and poly(fluoromethylmethacrylate) particles; inorganic materials such as metal chalcogenide, metal pnictide, silica, metal and metal oxide particles; metal oxides such as Al₂O₃, TiO₂, SnO₂, Sb₂O₅, Fe₂O₃, ZrO₂, CeO₂ and Y₂O₃; and metals such as gold, copper and silver,
   capable of forming a colloidal crystal that diffracts light having a wavelength in a range that corresponds to the wavelength of visible light, and
   0.0001-3 wt-%, preferably 0.0001-1 wt-%, more preferably 0.0001-0.5 wt-%, based on the total weight of the encapsulated monodisperse particles, of at least one broad spectrum absorber contrast agent and/or at least one precursor of a broad spectrum absorber contrast agent chosen from the group consisting of Alizarin Blue Black, Brilliant Blue Black, carbon black, black iron oxide, iron hydroxide, iron oxide black, silver oxide black, K, Ca, Sr, Ba, Zn, Pb, Fe, Ni, Ce, Co, Cr, Cu, Mn, Sn, Al, Ag, Mg, Au, Cd, Ag nitrate, Ag halogenide, Fe nitrate and Fe halogenide, which is encapsulated, with the proviso that the encapsulated monodisperse particle are not these of example 1 of PCT Application No PCT/EP2006/010882,
(ii) 30 wt-% and 99.99 wt-%, based on the total weight of the colourant composition, of at least one solvent, chosen from the group consisting of alcohols, esters, ketones, ethers and aliphatic and aromatic hydrocarbons having at least six carbon atoms and mixtures thereof including refinery distillation products and by-products.

A further embodiment of the present invention relates to a colourant composition (CC 11″) comprising
(i) 0.01 wt-% to 70 wt-%, based on the total weight of the colourant composition, of encapsulated monodisperse particles, comprising
   97-99.999 wt-%, preferably 99-99.9999 wt-%, more preferably 99.5-99.9999 wt-%, based on the total weight of the encapsulated monodisperse particles, of the monodisperse particle material chosen from the group consisting of organic polymer particles such as latex, acrylic, polystyrene, poly(vinyl acetate), polyacrylonitrile, poly(styrene-co-butadiene), polyester, polyamides, polyurethane, poly(methylmethacrylate) and poly(fluoromethylmethacrylate) particles; inorganic materials such as metal chalcogenide, metal pnictide, silica, metal and metal oxide particles; metal oxides such as Al₂O₃, TiO₂, SnO₂, Sb₂O₅, Fe₂O₃, ZrO₂, CeO₂ and Y₂O₃; and metals such as gold, copper and silver,
   capable of forming a colloidal crystal that diffracts light having a wavelength in a range that corresponds to the wavelength of visible light, and
   0.0001-3 wt-%, preferably 0.0001-1 wt-%, more preferably 0.0001-0.5 wt-%, based on the total weight of the encapsulated monodisperse particles, of at least one broad spectrum absorber contrast agent and/or at least one precursor of a broad spectrum absorber contrast agent chosen from the group consisting of Alizarin Blue Black, Brilliant Blue Black, carbon black, black iron oxide, iron hydroxide, iron oxide black, silver oxide black, K, Ca, Sr, Ba, Zn, Pb, Fe, Ni, Ce, Co, Cr, Cu, Mn, Sn, Al, Ag, Mg, Au, Cd, Ag nitrate, Ag halogenide, Fe nitrate and Fe halogenide, which is encapsulated, with the proviso that the encapsulated monodisperse particles are not consisting of methylmethacrylate and Alizarin Blue Black,
(ii) 30 wt-% and 99.99 wt-%, based on the total weight of the colourant composition, of at least one solvent, chosen from the group consisting of alcohols, esters, ketones, ethers and aliphatic and aromatic hydrocarbons having at least six carbon atoms and mixtures thereof including refinery distillation products and by-products.

A further embodiment of the present invention relates to a colourant composition (CC 12) comprising
(i) 0.01 wt-% to 70 wt-%, based on the total weight of the colourant composition, of encapsulated monodisperse particles, comprising
   97-99.999 wt-%, preferably 99-99.9999 wt-%, more preferably 99.5-99.9999 wt-%, based on the total weight of the encapsulated monodisperse particles, of the monodisperse particle material chosen from the group consisting of organic polymer particles such as latex, acrylic, polystyrene, poly(vinyl acetate), polyacrylonitrile, poly(styrene-co-butadiene), polyester, polyamides, polyurethane, poly(methylmethacrylate) and poly(fluoromethylmethacrylate) particles; inorganic materials such as metal chalcogenide, metal pnictide, silica, metal and metal oxide particles; metal oxides such as Al₂O₃, TiO₂, SnO₂, Sb₂O₅, Fe₂O₃, ZrO₂, CeO₂ and Y₂O₃; and metals such as gold, copper and silver,
   capable of forming a colloidal crystal that diffracts light having a wavelength in a range that corresponds to the wavelength of visible light, and
   0.0001-3 wt-%, preferably 0.0001-1 wt-%, more preferably 0.0001-0.5 wt-%, based on the total weight of the encapsulated monodisperse particles, of at least one broad spectrum absorber contrast agent and/or at least one precursor of a broad spectrum absorber contrast agent chosen from the group consisting of Alizarin Blue Black, Brilliant Blue Black, carbon black, black iron oxide, iron hydroxide, iron oxide black, silver oxide black, K, Ca, Sr, Ba, Zn, Pb, Fe, Ni, Ce, Co, Cr, Cu, Mn, Sn, Al, Ag, Mg, Au, Cd, Ag nitrate, Ag halogenide, Fe nitrate and Fe halogenide, which is encapsulated,
(iia) 30 wt-% and 99.99 wt-%, based on the total weight of the colourant composition, of water, and
(iib) optionally 0.1 wt-% and 89.99 wt-%, based on the total weight of the colourant composition of at least one solvent, chosen from the group consisting of alcohols, esters, ketones, ethers and aliphatic and aromatic hydrocarbons having at least six carbon atoms and mixtures thereof including refinery distillation products and by-products.

A further embodiment of the present invention relates to a colourant composition (CC 12′) comprising
(i) 0.01 wt-% to 70 wt-%, based on the total weight of the colourant composition, of encapsulated monodisperse particles, comprising
   97-99.999 wt-%, preferably 99-99.9999 wt-%, more preferably 99.5-99.9999 wt-%, based on the total weight of the encapsulated monodisperse particles, of the monodisperse particle material chosen from the group consisting of organic polymer particles such as latex, acrylic, polystyrene, poly(vinyl acetate), polyacrylonitrile, poly(styrene-co-butadiene), polyester, polyamides, polyurethane, poly(methylmethacrylate) and poly(fluoromethylmethacrylate) particles; inorganic materials such as metal chalcogenide, metal pnictide, silica, metal and metal oxide particles; metal oxides such as Al₂O₃, TiO₂, SnO₂, Sb₂O₅, Fe₂O₃, ZrO₂, CeO₂ and Y₂O₃; and metals such as gold, copper and silver,
   capable of forming a colloidal crystal that diffracts light having a wavelength in a range that corresponds to the wavelength of visible light, and
   0.0001-3 wt-%, preferably 0.0001-1 wt-%, more preferably 0.0001-0.5 wt-%, based on the total weight of the encapsulated monodisperse particles, of at least one broad spectrum absorber contrast agent and/or at least one precursor of a broad spectrum absorber contrast agent chosen from the group consisting of Alizarin Blue Black, Brilliant Blue Black, carbon black, black iron oxide, iron hydroxide, iron oxide black, silver oxide black, K, Ca, Sr, Ba, Zn, Pb, Fe, Ni, Ce, Co, Cr, Cu, Mn, Sn, Al, Ag, Mg, Au, Cd, Ag nitrate, Ag halogenide, Fe nitrate and Fe halogenide, which is encapsulated, with the proviso that the encapsulated monodisperse particles are not these of example 1 of PCT Application No PCT/EP2006/010882,
(iia) 30 wt-% and 99.99 wt-%, based on the total weight of the colourant composition, of water, and
(iib) optionally 0.1 wt-% and 89.99 wt-%, based on the total weight of the colourant composition of at least one solvent, chosen from the group consisting of alcohols, esters, ketones, ethers and aliphatic and aromatic hydrocarbons having at least six carbon atoms and mixtures thereof including refinery distillation products and by-products.

A further embodiment of the present invention relates to a colourant composition (CC 12″) comprising
(i) 0.01 wt-% to 70 wt-%, based on the total weight of the colourant composition, of encapsulated monodisperse particles, comprising
   97-99.999 wt-%, preferably 99-99.9999 wt-%, more preferably 99.5-99.9999 wt-%, based on the total weight of the encapsulated monodisperse particles, of the monodisperse particle material chosen from the group consisting of organic polymer particles such as latex, acrylic, polystyrene, poly(vinyl acetate), polyacrylonitrile, poly(styrene-co-butadiene), polyester, polyamides, polyurethane, poly(methylmethacrylate) and poly(fluoromethylmethacrylate) particles; inorganic materials such as metal chalcogenide, metal pnictide, silica, metal and metal oxide particles; metal oxides such as Al₂O₃, TiO₂, SnO₂, Sb₂O₅, Fe₂O₃, ZrO₂, CeO₂ and Y₂O₃; and metals such as gold, copper and silver,
   capable of forming a colloidal crystal that diffracts light having a wavelength in a range that corresponds to the wavelength of visible light, and
   0.0001-3 wt-%, preferably 0.0001-1 wt-%, more preferably 0.0001-0.5 wt-%, based on the total weight of the encapsulated monodisperse particles, of at least one broad spectrum absorber contrast agent and/or at least one precursor of a broad spectrum absorber contrast agent chosen from the group consisting of Alizarin Blue Black, Brilliant Blue Black, carbon black, black iron oxide, iron hydroxide, iron oxide black, silver oxide black, K, Ca, Sr, Ba, Zn, Pb, Fe, Ni, Ce, Co, Cr, Cu, Mn, Sn, Al, Ag, Mg, Au, Cd, Ag nitrate, Ag halogenide, Fe nitrate and Fe halogenide, which is encapsulated, with the proviso that the encapsulated monodisperse particles are not consisting of methylmethacrylate and Alizarin Blue Black
(iia) 30 wt-% and 99.99 wt-%, based on the total weight of the colourant composition, of water, and
(iib) optionally 0.1 wt-% and 89.99 wt-%, based on the total weight of the colourant composition of at least one solvent, chosen from the group consisting of alcohols, esters, ketones, ethers and aliphatic and aromatic hydrocarbons having at least six carbon atoms and mixtures thereof including refinery distillation products and by-products.

A further embodiment of the present invention relates to a colourant composition (CC 13) comprising
(i) 0.01 wt-% to 70 wt-%, based on the total weight of the colourant composition, of encapsulated monodisperse particles, comprising
   97-99.999 wt-%, preferably 99-99.9999 wt-%, more preferably 99.5-99.9999 wt-%, based on the total weight of the encapsulated monodisperse particles, of the monodisperse particle material chosen from the group consisting of organic polymer particles such as latex, acrylic, polystyrene, poly(vinyl acetate), polyacrylonitrile, poly(styrene-co-butadiene), polyester, polyamides, polyurethane, poly(methylmethacrylate) and poly(fluoromethylmethacrylate) particles; inorganic materials such as metal chalcogenide, metal pnictide, silica, metal and metal oxide particles; metal oxides such as Al₂O₃, TiO₂, SnO₂, Sb₂O₅, Fe₂O₃, ZrO₂, CeO₂ and Y₂O₃; and metals such as gold, copper and silver,
   capable of forming a colloidal crystal that diffracts light having a wavelength in a range that corresponds to the wavelength of visible light, and
   0.0001-3 wt-%, preferably 0.0001-1 wt-%, more preferably 0.0001-0.5 wt-%, based on the total weight of the encapsulated monodisperse particles, of at least one broad spectrum absorber contrast agent and/or at least one precursor of a broad spectrum absorber contrast agent chosen from the group consisting of Alizarin Blue Black, Brilliant Blue Black, carbon black, black iron oxide, iron hydroxide, iron oxide black, silver oxide black, K, Ca, Sr, Ba, Zn, Pb, Fe, Ni, Ce, Co, Cr, Cu, Mn, Sn, Al, Ag, Mg, Au, Cd, Ag nitrate, Ag halogenide, Fe nitrate and Fe halogenide, which is encapsulated,
(ii) 30 wt-% and 99.99 wt-%, based on the total weight of the colourant composition, of at least one solvent, chosen from the group consisting of alcohols, esters, ketones, ethers and aliphatic and aromatic hydrocarbons having at least six carbon atoms and mixtures thereof including refinery distillation products and by-products.

A further embodiment of the present invention relates to a colourant composition (CC 13′) comprising
(i) 0.01 wt-% to 70 wt-%, based on the total weight of the colourant composition, of encapsulated monodisperse particles, comprising
   97-99.999 wt-%, preferably 99-99.9999 wt-%, more preferably 99.5-99.9999 wt-%, based on the total weight of the encapsulated monodisperse particles, of the monodisperse particle material chosen from the group consisting of organic polymer particles such as latex, acrylic, polystyrene, poly(vinyl acetate), polyacrylonitrile, poly(styrene-co-butadiene), polyester, polyamides, polyurethane, poly(methylmethacrylate) and poly(fluoromethylmethacrylate) particles; inorganic materials such as metal chalcogenide, metal pnictide, silica, metal and metal oxide particles; metal oxides such as Al₂O₃, TiO₂, SnO₂, Sb₂O₅, Fe₂O₃, ZrO₂, CeO₂ and Y₂O₃; and metals such as gold, copper and silver,
   capable of forming a colloidal crystal that diffracts light having a wavelength in a range that corresponds to the wavelength of visible light, and
   0.0001-3 wt-%, preferably 0.0001-1 wt-%, more preferably 0.0001-0.5 wt-%, based on the total weight of the encapsulated monodisperse particles, of at least one broad spectrum absorber contrast agent and/or at least one precursor of a broad spectrum absorber contrast agent chosen from the group consisting of Alizarin Blue Black, Brilliant Blue Black, carbon black, black iron oxide, iron hydroxide, iron oxide black, silver oxide black, K, Ca, Sr, Ba, Zn, Pb, Fe, Ni, Ce, Co, Cr, Cu, Mn, Sn, Al, Ag, Mg, Au, Cd, Ag nitrate, Ag halogenide, Fe nitrate and Fe halogenide, which is encapsulated, with the proviso that the encapsulated monodisperse particles are not these of example 1 of PCT Application No PCT/EP2006/010882,
(ii) 30 wt-% and 99.99 wt-%, based on the total weight of the colourant composition, of at least one solvent, chosen from the group consisting of alcohols, esters, ketones, ethers and aliphatic and aromatic hydrocarbons having at least six carbon atoms and mixtures thereof including refinery distillation products and by-products.

A further embodiment of the present invention relates to a colourant composition (CC 13″) comprising
(i) 0.01 wt-% to 70 wt-%, based on the total weight of the colourant composition, of encapsulated monodisperse particles, comprising
   97-99.999 wt-%, preferably 99-99.9999 wt-%, more preferably 99.5-99.9999 wt-%, based on the total weight of the encapsulated monodisperse particles, of the monodisperse particle material chosen from the group consisting of organic polymer particles such as latex, acrylic, polystyrene, poly(vinyl acetate), polyacrylonitrile, poly(styrene-co-butadiene), polyester, polyamides, polyurethane, poly(methylmethacrylate) and poly(fluoromethylmethacrylate) particles; inorganic materials such as metal chalcogenide, metal pnictide, silica, metal and metal oxide particles; metal oxides such as Al₂O₃, TiO₂, SnO₂, Sb₂O₅, Fe₂O₃, ZrO₂, CeO₂ and Y₂O₃; and metals such as gold, copper and silver,
   capable of forming a colloidal crystal that diffracts light having a wavelength in a range that corresponds to the wavelength of visible light, and
   0.0001-3 wt-%, preferably 0.0001-1 wt-%, more preferably 0.0001-0.5 wt-%, based on the total weight of the encapsulated monodisperse particles, of at least one broad spectrum absorber contrast agent and/or at least one precursor of a broad spectrum absorber contrast agent chosen from the group consisting of Alizarin Blue Black, Brilliant Blue Black, carbon black, black iron oxide, iron hydroxide, iron oxide black, silver oxide black, K, Ca, Sr, Ba, Zn, Pb, Fe, Ni, Ce, Co, Cr, Cu, Mn, Sn, Al, Ag, Mg, Au, Cd, Ag nitrate, Ag halogenide, Fe nitrate and Fe halogenide, which is encapsulated, with the proviso that the encapsulated monodisperse particles are not consisting of methylmethacrylate and Alizarin Blue Black,
(ii) 30 wt-% and 99.99 wt-%, based on the total weight of the colourant composition, of at least one solvent, chosen from the group consisting of alcohols, esters, ketones, ethers and aliphatic and aromatic hydrocarbons having at least six carbon atoms and mixtures thereof including refinery distillation products and by-products.

A further embodiment of the present invention also relates to a colourant composition (CC 14) formulation which additionally comprises
(iii) at least one curing material, and
(iv) at least one initiator.

The colourant composition (CC 1), (CC 2), (CC 3), (CC 3′), (CC 3″) (CC 4), (CC 5), (CC 6), (CC 7), (CC 8), (CC 9), (CC 9′), (CC 9″), (CC 10), (CC 10′), (CC 10″), (CC 11), (CC 11′), (CC 11″), (CC 12), (CC 12′), (CC 12″), (CC 13), (CC 13′) and/or (CC 13″) according to the present invention can also comprises at least one curing agent and at least one initiator.

Any kind of commonly known curing agents can be used.

Usually, curing agents are resins which are crosslinkable. These are low molecular or oligomeric polyfunctional compounds with a molecular mass <1000 g/mol. The functional groups which are often terminal groups (for example epoxy-, isocyanate-, amine- or hydroxy-groups) are chosen that way (amount of groups as well as kind of the groups) that they react according to the polyaddition- or polycondensation-mechanism.

Suitable curing agents are epoxy acrylates, polyurethane acrylates, polyester acrylates, acrylated polyols and acrylated polyethers.

Such curing agents are used in an amount of 0.01 wt-%-15 wt-%, based on the total weight of the colourant composition. Preferably, curing agents are present in an amount of 0.1-10 wt-%, based on the total weight of the colourant composition.

In combination with the curing agent at least one initiator is used, which starts the polyaddition or polycondensation of the curing agent. This is usually done by light (400 nm-800 nm) or UV-light (100 nm-400 nm)

Such an initiator can be peroxide or peroxide containing compounds, benzophenone and benzophenone derivatives, acetophenone and acetophenone derivatives, benzoin ether derivatives, thioxanthones derivatives,

Such initiators are used in an amount of 0.005 wt-%-10 wt-%, based on the total weight of the colourant composition. Preferably, initiators are present in an amount of 0.01-8 wt-%, based on the total weight of the colourant composition.

A further embodiment of the present invention relates to a colourant composition (CC 15), which additionally comprises
(iii) 0.01 wt-% to 15 wt-%, based on the total weight of the colourant composition, of at least one curing material, and
(iv) 0.005 wt-% to 10 wt-%, based on the total weight of the colourant composition, of at least one initiator.

A further embodiment of the present invention relates to a colourant composition (CC 16), which additionally comprises
(iii) 0.01 wt-% to 15 wt-%, based on the total weight of the colourant composition, of at least one curing material chosen from the group consisting of epoxy acrylates, polyurethane acrylates, polyester acrylates, acrylated polyols and acrylated polyethers,
(iv) 0.005 wt-% to 10 wt-%, based on the total weight of the colourant composition, of at least one initiator chosen from the group consisting of peroxide or peroxide containing compounds, benzophenone and benzophenone derivatives, acetophenone and acetophenone derivatives, benzoin ether derivatives and thioxanthones derivatives.

All the preferences for the curing agent and the initiator in regard to the compounds as well as the concentrations can be applied to the colourant compositions (CC 1), (CC 2), (CC 3), (CC 3′), (CC 3″) (CC 4), (CC 5), (CC 6), (CC 7), (CC 8), (CC 9), (CC 9′), (CC 9″), (CC 10), (CC 10′), (CC 10″), (CC 11), (CC 11′), (CC 11″), (CC 12), (CC 12′), (CC 12″), (CC 13), (CC 13′) and/or (CC 13″) as described above as well.

Additionally the colouring composition CC 1), (CC 2), (CC 3), (CC 3′), (CC 3″) (CC 4), (CC 5), (CC 6), (CC 7), (CC 8), (CC 9), (CC 9′), (CC 9″), (CC 10), (CC 10′), (CC 10″), (CC 11), (CC 11′), (CC 11″), (CC 12), (CC 12′), (CC 12″), (CC 13), (CC 13′), (CC 13″), (CC 14), (CC 15) and/or (CC 16) can comprise further auxiliaries. Such auxiliaries are these commonly used in the field of colouring, such as coating and printing.

Auxiliaries are those additional chemicals which are used along with the colourant, to fix the colourants to the fabric or otherwise improve our results of the coloring process.

Furthermore, under the term auxiliaries is to be understood the chemicals, which help to improve the property of the formulation itself, such as storage, better manipulability of the colourant composition, etc.

Examples of auxiliaries are wetting agents, buffer substances, antistatic agents, bleaching agents, oxidation agents, rheology modifiers, solubilizers, siccative, antifoams, levelling agents, surfactants, electrolytes, foam suppressants, antifreezing agents or fungistatic and/or bacteriostatic agents, optical brighteners, softeners, flameproofing additives, or dirt repellents, water repellents and oil repellents, as well as water softeners and natural or synthetic thickeners, e.g. alginates and cellulose ethers.

Such auxiliaries are usually present in a smaller amount, which can go up to about 10 wt-%, based on the total weight of the colourant composition.

If one or more auxiliaries are present the amount goes usually from 0.1 wt-% to 10 wt-%, based on the total weight of the colourant composition.

Therefore a further embodiment of the present invention relates to a colourant composition as described above comprising additionally at least one auxiliary.

Therefore a further embodiment of the present invention relates to a colourant composition (CC 17) additionally comprising
(v) at least one auxiliary.

Therefore a further embodiment of the present invention relates to a colourant composition (CC 18) additionally comprising
(v) at least one auxiliary chosen from the group consisting of wetting agents, buffer substances, antistatic agents, bleaching agents, oxidation agents, rheology modifiers, solubilizers, siccative, antifoams, levelling agents, surfactants, foam suppressants, antifreezing agents or fungistatic and/or bacteriostatic agents, optical brighteners, softeners, flameproofing additives, or dirt repellents, water repellents and oil repellents, as well as water softeners and natural or synthetic thickeners, e.g. alginates and cellulose ethers.

Another embodiment of the present invention relates to a colourant composition (CC 19) additionally comprising
(v) 0.1 wt-% to 10 wt-%, based on the total weight of the colourant composition, of at least one auxiliary.

Another embodiment of the present invention relates to a colourant composition (CC 20) additionally comprising
(v) 0.1 wt-% to 10 wt-%, based on the total weight of the colourant composition, of at least one auxiliary from the group consisting of wetting agents, buffer substances, antistatic agents, bleaching agents, oxidation agents, rheology modifiers, solubilizers, siccative, antifoams, levelling agents, surfactants, foam suppressants, antifreezing agents or fungistatic and/or bacteriostatic agents, optical brighteners, softeners, flameproofing additives, or dirt repellents, water repellents and oil repellents, as well as water softeners and natural or synthetic thickeners, e.g. alginates and cellulose ethers.

All the preferences for the curing agent and the initiator in regard to the compounds as well as the concentrations can be applied to the colourant compositions CC 1), (CC 2), (CC 3), (CC 3′), (CC 3″) (CC 4), (CC 5), (CC 6), (CC 7), (CC 8), (CC 9), (CC 9′), (CC 9″), (CC 10), (CC 10′), (CC 10″), (CC 11), (CC 11′), (CC 11″), (CC 12), (CC 12′), (CC 12″), (CC 13), (CC 13′), (CC 13″) (CC 14), (CC 15) and/or (CC 16) as described above as well.

As already mentioned the colourant compositions according to the present invention can be in any suitable physical form. Usually it is in the form of a liquid, a gel, mousse, wax or a paste.

A further embodiment of the present invention relates to a colourant composition as described above, which is a coating and/or a printing formulation.

The printing process can be done according to any well known processes such as Ink jet (such as Bubble Jet, Compound jet, Dry Inkjet, Hotmelt Inkjet), relief printing, intaglio, letterpress, lithography, flexography, gravure, screen printing and pad printing. Therefore the formulations have to be adapted to the desired form of printing technology. Formulations for the inkjet technology comprising monodisperse particles are for example known from WO2005/063902

Therefore a further embodiment of the present invention relates to a colourant composition which is a printing formulation for Ink jet (such as Bubble Jet, Compound jet, Dry Inkjet, Hotmelt Inkjet), relief printing, intaglio, letterpress, lithography, flexography, gravure, screen printing and pad printing.

The colourant composition as described in the present patent application can also be used for any known coating technology.

Therefore the formulations have to be adapted to the desired form of coating technology.

Suitable coating processes are for example knife coating, immersion (dip) coating, Gap Coating, Curtain coating, rotary screen, Reverse Roll coating, Gravure coating, Metering rod (Meyer bar) coating, Slot Die (Extrusion) coating and Hot Melt coating.

The encapsulated monodisperse particles, the colloidal crystals and/or the colourant compositions according to the present invention can also be used in personal care formulations especially cosmetic formulations.

Therefore a further embodiment of the present invention is the use of monodisperse particles as described above and/or colloidal crystal as described above and/or colourant compositions as described above in personal care formulations and/or cosmetic formulations.

Therefore a further embodiment of the present invention also relates to personal care formulation and/or cosmetic formulation comprising monodisperse particles as described above and/or a colloidal crystal as described above and/or at least one colourant composition as described above.

The personal care formulations and/or cosmetic formulations can have any usual form of application. They can be in the form of, for example, solutions, suspensions, emulsions, PIT emulsions, pastes, ointments, gels, creams, lotions, powders, soaps, surfactant-containing cleansing preparations, oils, aerosols, sprays, mousse and sticks.

The personal care formulations and/or cosmetic formulations can be used for any personal care and/or cosmetic application. They can be used for example as lipsticks, lip-cares sticks, mascara, eyeliner, eye-shadow, rouge, powder make-up, emulsion make-up, wax make up, nail lacquer, shampoos and shower compositions.

The personal care formulations as well as the cosmetic formulations may comprise any commonly used ingredients and additives for a cosmetic use, such as solvents, further dyes and/or pigments, antioxidants, repellents, vitamins, UV-absorbers, solutes, self-tanning agents, preservatives, antioxidants, stabilisers, solubilisers, vitamins, colorants and odour improvers.

Preferred cosmetic compositions are those suitable for the application to human skin, which optionally, but preferably, include a skin benefit agent in addition to the colourant compositions of the present invention. Suitable additional skin benefit agents include anti-aging, wrinkle-reducing, skin whitening, anti-acne and sebum reduction agents. Examples of these include alpha-hydroxy acids, beta-hydroxy acids, polyhydroxy acids, hydroquinone, t-butyl hydroquinone, Vitamin B and C derivatives, dioic acids, retinoids; betulinic acid; vanillic acid; allantoin, a placenta extract; hydrolactin; and resorcinol derivatives.

Following a suitable contact time, excess composition can be removed/washed off if necessary. Preferably the cosmetic composition is in contact with the skin, nail or hair for sufficient time such that at least two or three colloidal crystalline layers are formed.

The concentration of the ingredients can vary a lot, but a person skilled in the art knows which concentration of a specific ingredient is necessary to produce the various application forms.

The new encapsulated monodisperse particles according to the present invention can be used in any known personal care formulations and cosmetic formulations. Suitable formulations can be found for example in US2006002875.

Therefore a further embodiment of the present invention is a personal care formulation comprising encapsulated monodisperse particles according to the present invention.

Therefore a further embodiment of the present invention is a cosmetic formulation comprising encapsulated monodisperse particles.

A further area of application for the encapsulated monodisperse particles is the security sector with various applications, for example in bank notes, credit cards, visas, for tax seals or the like.

Therefore a further embodiment of the present invention is a security printing and/or coating ink comprising encapsulated monodisperse particles according to the present invention.

Such an ink is used to produce security marking, thread or device, hologram, hot stamping foil or watermark, in particular for the purpose of prevention of counterfeiting, authentification, verification, or identification of data or information, comprising an optically variable marking as described above and below.

The security elements also comprise an additional detectable security feature, in particular an optically, machine or haptically detectable security feature.

Therefore a further embodiment of the present invention is a security printing and/or coating ink comprising encapsulated monodisperse particles as described above and at least one additional detectable security feature, in particular an optically, machine or haptically detectable security feature.

Optically detectable security features are those which can be detected by without using an apparatus or with the help of a simple apparatus.

Machine detectable security features are those which can be detected by using an apparatus able to detect luminous, magnetic, electrically conductive, thermoelectronical or piezoelectronical properties.

Haptically detectable security features are those which can be detected by the human sense of touch.

In WO2006/045567 there can be found compounds which are added to obtain these additional detectable security features.

The monodisperse particles and/or the colloid crystals and/or the colouring compositions can also be used for home care applications. The particles, crystals and/or compositions are used usually to give the product a specific visual appearance.

The colourant compositions of the present invention may be applied to any suitable substrate to colour at least a region of the substrate. A structural colour effect is produced due to direct reflection and/or diffraction of light in the wavelength of visible light by the colloidal crystal. Substantially all of the light that is diffused by the colloidal crystal is absorbed by the broad spectrum absorber contrast agent. This causes an enhancement of the structural colour effect. The substrate to be coloured can have any possible form as well as size.

As discussed above, when the colourant composition comprises monodisperse particles capable of forming a colloidal crystal, the colloidal crystal typically is formed as the colourant composition is applied to a substrate. The colourant compositions may be applied to a substrate by any suitable method, for example by painting, spraying and/or washing. When the colourant composition comprises a colloidal crystal formed from monodisperse particles, the colloidal crystal similarly is deposited on a substrate by any suitable method.

According to one aspect of the present invention, there is provided a substrate which comprises at least one colloidal crystalline layer, which colloidal crystalline layer comprises (i) a colloidal crystal formed from monodisperse particles and (ia) at least one broad spectrum absorber contrast agent and/or at least one precursor of a broad spectrum absorber contrast agent, and wherein the colloidal crystalline layer forms a colour on at least a region of the substrate, for example in the form of letters, numbers and/or other symbols, and/or a graphic design.

According to another aspect of the present invention, there is provided a substrate which comprises at least one colloidal crystalline layer, which colloidal crystalline layer comprises (i) a colloidal crystal formed from monodisperse particles and (ii) at least one organic broad spectrum absorber contrast agent, and/or at least one precursor of a broad spectrum absorber contrast agent and wherein the colloidal crystalline layer forms a colour on at least a region of the substrate, for example in the form of letters, numbers and/or other symbols, and/or a graphic design.

In the colloidal crystalline layer, the monodisperse particles, colloidal crystal and broad spectrum absorber contrast agent are as hereinbefore defined.

In the colloidal crystalline layer, the broad spectrum absorber contrast agent and/or at least one precursor of a broad spectrum absorber contrast agent is encapsulated in the monodisperse particles and positioned in interstices between the monodisperse particles in the colloidal crystal and/or located on the surface of the monodisperse particles. In one aspect, in the colloidal crystalline layer the broad spectrum absorber contrast agent and/or at least one precursor of a broad spectrum absorber contrast agent is present by being encapsulated in the monodisperse particles only.

Preferred substrates are those with surface irregularities that act as sites for crystal nucleation. Substrates include fibre (such as hair), skin, nails, food material, stone, ceramic, glass, paper, fabrics, wood, leather, metal (for example aluminium) and plastics. The object to be coated can also be a combination of various substrates and it can have any form.

The coating and/or printing formulations according to the present invention are very suitable to colour (completely or in parts) packaging, which are for example used to sell commercial products, such as toothpaste containers, cans for drinks, shampoo container, shower gel container etc.

The colourant compound can also be used to print labels, which are then put onto a specific embodiment.

According to another aspect of the present invention, there is provided the use of a colourant composition as hereinbefore defined for colouring a substrate.

According to another aspect of the present invention, there is provided a method of colouring a substrate, which method comprises the step of contacting at least a region of the substrate with a colourant composition as hereinbefore defined, such that a colloidal crystalline layer forms on the substrate.

As the skilled person would appreciate, the colloidal crystalline layer typically forms on the region of the substrate that is contacted with the colourant composition. It is not essential for the whole of the substrate to be contacted with the colourant composition. In other words, the coverage of colloidal crystalline layers need not be complete, i.e. it can be discontinuous.

By the term “colloidal crystalline layer” we mean a sheet or thickness of material comprising colloidal crystals and contrast agent, for example covering at least a region of a suitable substrate. Depending on the nature of the substrate, which may be porous, colloidal crystalline layers may form on the surface of, and/or within, the substrate. Furthermore, the colloidal crystalline layer(s) need not be entirely regular, provided that the desired structural colour effects are achieved. In other words, some crystal disorder is permitted.

According to another aspect of the present invention, there is provided a colloidal crystalline layer which comprises (i) a colloidal crystal formed from monodisperse particles and (ii) at least one broad spectrum absorber contrast agent and/or at least one precursor of a broad spectrum absorber contrast agent, which is encapsulated in the monodisperse particle and wherein the colloidal crystalline layer forms a colour.

There is also provided a method of forming a colloidal crystalline layer on a substrate, which method comprises the step of applying a colourant composition as hereinbefore defined to at least a region of the substrate.

In the colloidal crystalline layer, the colloidal crystals reflect and/or diffract light having a wavelength in a range that corresponds to the wavelength of visible light, so as to provide a structural colour effect. Any visible light that is diffused by the colloidal crystals is substantially all absorbed by the broad spectrum absorber contrast agent. This causes an enhancement of the structural colourant effect, as discussed above.

Suitable substrates include any substrate upon which a colloidal crystalline layer may form. Suitable substrates include, for example, fibre (such as hair), skin, nails, food material, stone, ceramic, glass, paper, fabrics, wood, leather, metal (for example aluminium) and plastics.

When the substrate is a food material, the colourant composition must be of a grade that can be used in food materials. Food materials in which the colourant compositions of the present invention may be used include, for example, eggs, fruit, vegetables, ice creams, sauces, water ice and chocolate.

For examples when vegetables or fruits are used, it is possible to coat the parts and/or print onto the parts which can be eaten as well as the parts, which are (usually) not eaten, like the peel, leaves, etc.

When the substrate is a plastics material, the colourant composition of the present invention may be dispersed in the plastics material, which may then be moulded for example by injection moulding, injection blow moulding or blow moulding.

The substrate may further comprise a protective material, for example as a protective covering or coating. The protective covering or coating may, for example, comprise a clear lacquer layer on a surface of the colloidal crystalline layer. Alternatively, the protective material may be formed in situ, for example by providing monodisperse particles having appropriately modified surfaces.

Thus, according to one aspect of the present invention, there is provided the use of a colourant composition as hereinbefore defined for colouring the hair of an individual.

There is also provided a method of colouring the hair of an individual which method comprises the step of contacting at least a region of the hair of the individual with a colourant composition as hereinbefore defined such that a colloidal crystalline layer forms on the hair.

There is also provided a hair which comprises at least one colloidal crystalline layer, which colloidal crystalline layer comprises (i) a colloidal crystal formed from monodisperse particles and (ii) at least one broad spectrum absorber contrast agent and/or at least one precursor of a broad spectrum absorber contrast agent, which is encapsulated in the monodisperse particle and wherein the colloidal crystalline layer forms a colour on at least a region of the hair.

There is also provided a hair dye composition comprising a colourant composition as hereinbefore defined.

The hair dye compositions of the present invention may be in any suitable form. For example, the hair dye compositions may be in the form of sprays, lotions, shampoos, creams or pastes which can be applied directly to all or part of the hair. Following a suitable contact time, excess composition can then be washed off if necessary. Preferably the hair dye composition is in contact with the hair for sufficient time such that at least two or three colloidal crystalline layers are formed.

According to another aspect of the present invention, there is provided the use of a colourant composition as hereinbefore defined for colouring a fabric.

Colouring of fabrics includes the ‘brightening’ of fabrics, such in the case of white textile materials.

According to another aspect of the present invention, there is provided a method of colouring a fabric which method comprises the step of contacting at least a region of the fabric with a colourant composition as hereinbefore defined such that a colloidal crystalline layer forms on the fabric.

Colorant compositions for use in colouring fabrics can be applied as part of standard laundry formulations known in the art such as powders or tablets that dissolve/disperse in water or as liquids.

According to another aspect of the present invention, there is provided a fibrous material which comprises at least one colloidal crystalline layer, which colloidal crystalline layer comprises (i) a colloidal crystal formed from monodisperse particles and (ia) at least one broad spectrum absorber contrast agent and/or at least one precursor of a broad spectrum absorber contrast agent, which is encapsulated in the monodisperse particles and wherein the colloidal crystalline layer forms a colour on at least a region of the fibrous material.

The fibrous material may, for example, be a fabric. Suitable fabrics include natural and synthetic fabrics. Examples of natural fabrics include wool, silk, fur, cellulosic materials such as cotton, flax, linen and hemp. Synthetic fabrics include, for example, viscose, nylon (polyamide), acrylic (polyacrylonitrile), aramid (aromatic polyamide) and polyester. The fabric may be in any suitable form, for example woven, non-woven or knitted.

According to another aspect of the present invention, there is provided a fabric dye composition comprising a colourant composition as hereinbefore defined.

Preferably the fabric dye composition is in contact with the fibrous material for sufficient time such that at least two or three colloidal crystalline layers are formed.

The fabric dye composition of the present invention may be in any suitable form. For example, the fabric dye composition may be in the form of a solid, liquid or paste.

According to another aspect of the present invention, there is provided the use of a colourant composition as hereinbefore defined for colouring paper.

According to another aspect of the present invention, there is provided a method of colouring paper which method comprises the step of contacting at least a region of the paper with a colourant composition as hereinbefore defined such that a colloidal crystalline layer forms on the paper.

By the term “paper” we mean any material that is manufactured in sheets from the pulp of wood or other fibrous substances and that is manufactured for any use, including for example writing or printing on, wrapping or packaging.

According to another aspect of the present invention, there is provided a paper product which comprises at least one colloidal crystalline layer, which colloidal crystalline layer comprises (i) a colloidal crystal formed from monodisperse particles and (ia) at least one broad spectrum absorber contrast agent and/or at least one precursor of a broad spectrum absorber contrast agent, which is encapsulated in the monodisperse particle and wherein the colloidal crystalline layer forms a colour on at least a region of the paper. Preferably, the colour is formed in the shapes of letters, numbers and/or other symbols, and/or a graphic design on the paper.

According to another aspect of the present invention, there is provided an ink composition comprising a colourant composition as hereinbefore defined. For example, the ink composition is suitable for printing on a printable surface such as paper or fabric.

Ink compositions of the present invention can typically be applied to a substrate using standard printing techniques known in the art for applying inks to a range of substrates. Typically, the ink compositions are applied to the substrate to form letters, numerals and/or other symbols, and/or graphic designs.

In the above applications/uses, it is sufficient for a single colloidal crystalline layer to form on or within the substrate. However, it is preferred that at least two or three colloidal crystalline layers are formed. As discussed above, the colloidal crystalline layers may be complete or discontinuous and may form on the surface of, and/or within, the substrate.

When the colourant compositions of the present invention are used to colour natural substrates, such as hair, nail, tooth and natural fabrics such as wool or cotton, the compositions typically comprise from 0.01 to 2% (preferably from 0.01 to 1%) by weight of the monodisperse particles or colloidal crystals and from 0.01 to 2% (preferably from 0.01 to 1%) by weight of the broad spectrum absorber contrast agent and/or at least one precursor of a broad spectrum absorber contrast agent. When the colourant compositions of the present invention are used to colour synthetic substrates, the compositions typically comprise from 0.5 to 15% (preferably from 1 to 10%) by weight of the monodisperse particles or colloidal crystals and from 0.5 to 15% (preferably from 1 to 10%) by weight of the broad spectrum absorber contrast agent and/or at least one precursor of a broad spectrum absorber contrast agent.

As the skilled person would appreciate, the hair dye, cosmetic, fabric dye and ink compositions discussed above preferably do not contain any additional ingredients that could prevent and/or discourage the formation of colloidal crystals in use.

The present invention will now be described further with reference to the following non-limiting examples.

### EXAMPLE 1

### Synthesis of Monodisperse Poly(methylmethacrylate) Particles Encapsulating a Dye

A three necked flask (250 ml) was charged with 150 ml of double deionised water and 4 to 40 mg of a dye (for example Brilliant Black BN). The flask was sealed with a septum. The flask was heated up to 90° C. and flushed with nitrogen for 45 minutes. After the nitrogen flow had stopped, 15 ml (141 mmol) of methylmethacrylate was added through the septum. The polymerisation was initiated with potassium peroxodisulfate after another 30 minutes at 90° C. by adding 5 ml (1.8 mmol, 500 mg) of a 10% by weight solution. The solution was flushed for 10 minutes at 90° C. with nitrogen. The reaction solution was stirred with a mechanical quirler at 400 revolutions per minute (rpm). After 2.5 hours reaction time, the flask was opened and the resulting warm solution was filtered through a standard paper filter to remove large agglomerations. The solution was then washed twice for 5 to 10 minutes in the centrifuge at 4000 rpm to separate the transparent pellet. Afterwards it was centrifuged for 30 to 90 minutes, until a clear liquid was formed above the iridescent pellet. The liquid was then poured out and the pellet was re-dispersed in 60 ml of double deionised water. This was repeated for another 4 times in order to separate the polymer from oligomers. The solution were then stored as a 10 to 20% by weight suspension. The yield depended on the separated polymer/oligomer ratio and was typically in a range of from about 50 to 90%.

The polymer spheres grown by this method showed surface charges, which give rise to electrostatic repulsion that prevent aggregation.

The monodisperse dye encapsulated polymer spheres produced had average particle diameters of between about 0.05 and 0.7 μm, with a standard deviation of less than 4%.

The samples were then purified by centrifuging the dispersion at 5000 rpm for 20 minutes to separate the solid from the liquid. The solid was re-dispersed in distilled water to the original volume by mechanical stirring and ultrasonic treatment. This procedure was repeated three or four times.

The samples produced by this method exhibited a brilliant colour due to Bragg diffraction of visible light.

### EXAMPLE 2

### Fabrication of Colloidal Crystals in a Test-Tube

A suspension of 10% of uniform-sized poly(methylmethacrylate) spheres (prepared as Example 1) in water (particle volume fraction: 10%; diameter: 210 nm; standard deviation: 3%) containing volume fraction of 0.3% Brilliant Black BN was poured in a 2 ml plastic vial (8 mm wide and 30 mm long) and then centrifuged at 10000 rpm (about 3500 G) for 10 minutes. After centrifugation, an iridescent crystalline region formed at the bottom of the cell, a turbid noncrystalline region in the middle, and a transparent region on the top.

The same method was also performed on a solution containing bare poly(methylmethacrylate) particles (particle volume fraction: 10%; diameter: 210 nm; standard deviation: 3%) in distilled water (with no dye, pigment and/or chromophore). The colloidal crystal formation was performed under the same conditions as those described above. After centrifugation, an iridescent crystalline region formed at the bottom of the cell, a turbid noncrystalline region in the middle, and a transparent region on the top.

1. Monodisperse particles capable of forming a colloidal crystal, the monodisperse particles consisting of only one layer and not being core-shell particles, wherein at least one broad spectrum absorber contrast agent and/or at least one precursor of a broad spectrum absorber contrast agent is encapsulated in the particles, characterised in that the monodisperse particles comprise
(i) 97-99.999 wt-%, based on the total weight of the monodisperse particles, of the monodisperse particle material, and
(ii) 0.0001-3 wt-%, based on the total weight of the monodisperse particles, of at least one broad spectrum absorber contrast agent and/or at least one precursor of a broad spectrum absorber contrast agent, which is encapsulated so as to be distributed on the whole of the monodisperse particles.

2. Monodisperse particles according to claim 1 wherein,
(i) 99-99.999 wt-%, based on the total weight of the monodisperse particles, of the monodisperse particles material and
(ii) 0.0001-1 wt-%, based on the total weight of the monodisperse particles, of at least one broad spectrum absorber contrast agent and/or at least one precursor of a broad spectrum absorber contrast agent.

3. Monodisperse particles according to claim 1 wherein,
(i) 99.5-99.9999 wt-%, based on the total weight of the monodisperse particles, of the monodisperse particles material and
(ii) 0.0001-0.5 wt-%, based on the total weight of the monodisperse particles, of at least one broad spectrum absorber contrast agent and/or at least one precursor of a broad spectrum absorber contrast agent.

4. Monodisperse particles according to claim 1, wherein monodisperse particles consisting of methylmethacrylate and Alizarin Blue Black are excluded from the scope of protection.

5. Monodisperse particles according to claim 1, wherein the broad spectrum absorber contrast agent and/or at least one precursor of a broad spectrum absorber contrast agent is a mixture of compounds and wherein that mixture has the same absorption property as a single broad spectrum absorber contrast agent.

6. Monodisperse particles according to any of the preceding claims,
wherein the monodisperse particles are capable of forming a colloidal crystal that reflects and/or diffracts light having a wavelength in a range that corresponds to the wavelength of visible light.

7. Monodisperse particles according to claim 1, characterised in that they have a rms diameter of less than about 1 μm and greater than about 1 nm.

8. (canceled)

9. (canceled)

10. Monodisperse particles according to claim 1, characterised in that they have a rms diameter in the range of from 120 nm to 800 nm.

11. Monodisperse particles according to claim 1 characterised in that the particles have the shape of needles, plates, rods or spheres.

12. Monodisperse particles according to claim 1 wherein the broad spectrum absorber contrast agent is distributed over the whole monodisperse particle.

13. Monodisperse particles according to claim 1 characterised in that the monodisperse particles are made from organic and/or inorganic materials.

14. Monodisperse particles according to claim 13, characterised in that the monodisperse particles are made from organic polymer particles selected from the group consisting of latex, polystyrene, polyvinyl acetate), polyacrylonitrile, poly(styrene-co-butadiene), polyester, polyamides, polyurethane, poly(methylmethacrylate) and poly(fluoromethylmethacrylate) particles.

15. Monodisperse particles according to claim 11, characterised in that the monodisperse particles are made from metal chalcogenide, metal pnictide, silica, metal and metal oxide particles.

16. Monodisperse particles according to claim 1 wherein the broad spectrum absorber contrast agent is organic.

17. Monodisperse particles according to claim 1 wherein the broad spectrum absorber contrast agent is inorganic.

18. Monodisperse particles according to, claim 1 wherein the broad spectrum absorber contrast agent is chosen from the group consisting of Ag, Alizarin Blue Black, Brilliant Blue Black, carbon black, iron hydroxide and iron oxide black.

19. Monodisperse particles according to claim 1, wherein the precursor broad spectrum absorber contrast agent is a metal salt or a mixture of metal salts, wherein the metal can be chosen from K, Ca, Sr, Ba, Zn, Pb, Fe, Ni, Ce, Co, Cr, Cu, Mn, Sn, Al, Ag, Mg, Au, and Cd.

20. Monodisperese particles according to claim 19, wherein the metal salts are hydrophilic.

21. Monodisperese particles according to claim 19, wherein the metal salts are AgNO₃, FeCl₂ and FeCl₃.

22. (canceled)

23. A colourant composition used in a coating and/or printing formulation, a personal care formulation, a security printing or coating ink, or for printing and/or coating a substrate, said composition comprising
(i) monodisperse particles according to claim 1 and/or at least one colloidal crystal formed from the monodisperse particles according to claim 1, and
(ii) optionally, monodisperse particles, wherein no broad spectrum absorber contrast agent and no precursor of a broad spectrum absorber contrast agent is encapsulated.

24. (canceled)

25. Colourant composition according to claim 23 additionally comprising at least one broad spectrum absorber contrast agent and/or at least one precursor of a broad spectrum absorber contrast agents, which is not encapsulated by the monodisperse particles.

26. Colourant composition according to claim 25 wherein the additional broad spectrum absorber contrast agent and/or the precursor of a broad spectrum absorber contrast agent is positioned in interstices between the encapsulated monodisperse particles in the colloidal crystal.

27. Colourant composition according to claim 25, wherein the additional broad spectrum absorber contrast agent and/or at least one precursor of a broad spectrum absorber contrast agent is positioned on the surface of the encapsulated monodisperse particles.

28. (canceled)

29. (canceled)

30. Colourant composition according to claim 25, wherein the broad spectrum absorber contrast agent is chosen from the group consisting of Alizarin Blue Black, Brilliant Blue Black, carbon black, iron hydroxide and iron oxide black.

31. Colourant composition according to claim 25, wherein the precursors of broad spectrum absorber contrast agent is chosen from the group consisting AgNO₃, FeCl₂ and FeCl₃.

32. (canceled)

33. Colourant composition according to claim 23 wherein the amount of the monodisperse particles wherein at least one broad spectrum absorber contrast agent and/or at least one precursor of a broad spectrum absorber contrast agent is encapsulated therein is from 0.01 wt-% to 70 wt-%, based on the total weight of the colouring composition.

34-36. (canceled)

37. Colourant composition according to claim 23 comprising
(iiia) water, and/or
(iiib) at least one solvent, chosen from the group consisting of alcohols, esters, ketones, ethers and aliphatic and aromatic hydrocarbons having at least six carbon atoms and mixtures thereof including refinery distillation products and by-products.

38. (canceled)

39. Colourant composition according to claim 37 wherein the amount of solvent is in the range of 10 wt-% to 99.99 wt-%, preferably from 20 wt-% to 99.9 wt-%, and more preferably from 30 wt-% to 99.9 wt-%, based on total weight of the colourant composition.

40. Colourant composition according to claim 37 wherein the colourant composition is a liquid, gel, mousse, wax or a paste.

41. (canceled)

42. Colourant composition according to claim 23 comprising additionally
(iii) at least one curing agent is selected from the group consisting of epoxy acrylates, polyurethane acrylates, polyester acrylates, acrylated polyols and acrylated polyethers, and
(iv) at least one initiator selected from the group consisting of peroxide or peroxide containing compounds, benzophenone and benzophenone derivatives, acetophenone and acetophenone derivatives, benzoin ether derivatives and thioxanthones derivatives.

43-46. (canceled)

47. Colourant composition according to claim 23 additionally comprising
(v) at least one auxiliary chosen from the group consisting of wetting agents, buffer substances, antistatic agents, bleaching agents, oxidation agents, rheology modifiers, solubilizers, siccative, antifoams, levelling agents, surfactants, foam suppressants, antifreezing agents or fungistatic and/or bacteriostatic agents, optical brighteners, softeners, flameproofing additives, or dirt repellents, water repellents and oil repellents, as well as water softeners and natural or synthetic thickeners, e.g. alginates and cellulose ethers.

48-65. (canceled)
